(19) United States
(12) Patent Application Publication
(10) Pub. No.: US 2009/0105826 A1
(43) Pub. Date: Apr. 23, 2009

(54) Surgical Implants

(76) Inventors: Alan McLeod, Somerset (GB); Christopher Reah, Taunton (GB)

Correspondence Address: JONATHAN SPANGLER;NuVasive, Inc., 7475 LUSK BOULEVARD, SAN DIEGO, CA, 92121, US

(21) Appl. No.: 11/921,542

(22) PCT Filed: Jun. 5, 2006
(86) PCT No.: PCT/US2006/021814

§ 371 Date: Dec. 3, 2007

(30) Foreign Application Priority Data
Jun. 3, 2005 (GB) 0511329.5
Jul. 20, 2005 (GB) 051489.1

(51) Int. Cl. A61F 2/44; A61B 17/04; A61B 19/00

(52) U.S. Cl. 623/17.16; 606/300; 128/898

(57) ABSTRACT

Disc prostheses, particularly for use in the lumbar region of the spine are provided. The disc prosthesis includes a core comprising one or more filling elements provided within an inner component of fabric. The inner component is provided within an outer component of fabric. By providing a smooth inner contact surface between the inner component and the core filling, movement between the inner and outer components is facilitated in preference to movement between the inner component and core. Core abrasion is thus avoided. The use of an inner component and an outer component also means that the characteristics of each can be optimised to meet different aims.

### CROSS REFERENCE TO RELATED APPLICATIONS

The present invention is an International Patent Application and claims the benefit of priority from commonly owned and co-pending British Patent Application No. 0511329.5, entitled “Improvements Relating in and to Surgical Implants” and filed on Jun. 3, 2005, and commonly owned and co-pending British Patent Application No. 0514891.1, entitled “Improvements Relating in and to Implants” and filed Jul. 20, 2005, the entire contents of which are hereby expressly incorporated by reference into this disclosure as if set forth in their entirety herein.

### BACKGROUND OF THE INVENTION

I. Field of the Invention

This invention concerns improvements in and relating to surgical implants, particularly, but not exclusively in relation to surgical implants for the replacement of intervertebral discs, particularly, but not exclusively in the lumbar region of the spine.

II. Discussion of the Prior Art

Increasingly there is a desire to address problems with intervertebral discs by replacing all or part of the disc with a prosthetic disc rather than fusing the adjacent vertebrae. A wide variety of designs of disc prostheses exist. Generally they are based upon either articulated metal plates or metal end plates with a polyethylene spacer. Generally such devices face problems in terms of the reduced mobility they provide, are reliant upon absolutely correct positioning and do not emulate fully the normal motion they aim to replace.

Previously there has been developed a disc prosthesis including an element of elastomeric or visco-elastic material, the element being provided in a retaining fabric, U.S. Pat. No. 6,093,205. The disk prosthesis was particularly developed for use in the cervical region of the spine.

### SUMMARY OF THE INVENTION

The present invention has amongst its aims to provide an improved partial or total spinal disc replacement, particularly in the lumber region. The present invention has amongst its aims to provide a more reliable spinal disc replacement, particularly for the lumbar region.

According to a first aspect of the present invention we provide a disc prosthesis including a core of one or more filling elements, the core being provided within an inner component, the inner component being provided within an outer component.

Various options, possibilities and features for the first aspect of the invention are now provided.

The core may be formed of multiple filling elements. Multiple filling element forms for the core are particularly suited to minimally invasive surgical techniques as the core can be formed in the inner and/or outer component in-situ.

The core and inner component may be formed of different materials and/or formed in different ways and/or be provided with different properties. In particular the core may mimic the properties of the nucleus and the inner component may mimic the properties of the annulus, or properties intermediate the nucleus and annulus. The outer component may be provided with one or more parts, potentially integral therewith or attached thereto, which form the inner component. The core and/or inner component and/or outer component in such an embodiment may be formed of different materials and/or formed in different ways and/or be provided with different properties. In particular the core may mimic the properties of the nucleus and/or the inner component may mimic the properties of the annulus, or properties intermediate the nucleus and annulus and/or the outer component may mimic properties of the annulus and/or the anterior longitudinal ligament(s).

The core may be formed of a single material type or of multiple material types. Preferably the core may be formed of one or more fibrous filing elements. Alternatively, the core may be formed of a plurality of elastomeric and/or viscoelastic filling elements.

The plurality of elastomeric and/or viscoelastic filing elements may be formed of material including, but not necessarily limited to hydrogel and silicone based filling elements having a shore hardness of 35 to 80°, and the filling elements may be impregnated and/or doped and/or provided with further materials including, but not necessarily limited to barium sulphate.

The core may be provided of one or more fibrous filling elements, for instance such material may be provided in a single plane. The fibrous material may be provided with a proportion, preferably the majority, of the fibres at an angle of between 10 and 80 degrees to the horizontal. Such a material may be provided of embroidery and/or other fibrous assembly technique. Preferably such a material resembles the structure and/or properties of the fibrous material of the spine. The core may be formed of coiled filling element(s), particularly a fibrous material. Such a fibrous material may be elastomeric and/or polyester and/or the other fibre materials mentioned herein.

One or more filling elements may include and/or be formed from one or more fibre materials. One or more of the one or more filling elements may include and/or be formed of one or more of polyester, polypropylene, polyethylene, glass fibre, glass, polyaramide, metal, copolymers, polylactic acid, polyglycolic acid, biodegradable materials, silk, cellulose or polycaprolactone.

Preferably one or more filling elements that are porous and/or define voids within themselves and/or between parts of a filling element are provided. The pores and/or voids and/or apertures and/or gaps provided in or by the filling elements ideally provide fluid communication through the filling elements and/or there between. Preferably a large number of pores and/or voids and/or apertures and/or gaps are provided in the material from which filling elements are formed. Preferably a large number of pores and/or voids and/or apertures and/or gaps are provided by one or more of the filling elements. Preferably a large number of pores and/or voids and/or apertures and/or gaps are provided within one or more of the filling elements by virtue of their structure.

One or more filling elements may be formed of unconstrained fibres. One or more filling elements may be formed of unbraided fibres. One or more filling elements of felt or felt-like material may be provided. One or more filling elements with interlaced fibres may be provided. One or more filling elements may be provided with aligned fibres. One or more filling elements may be provided with one or more groups of aligned fibres and/or one or more non-aligned fibres and/or one or more groups of fibres on different alignments to the first. One or more filling elements with non-linear fibres may be provided. One or more filling elements with wavy and/or curved and/or zig zag fibres may be provided. One or more filling elements with fibres which act to space each other from one another may be provided. One or more filling elements with primary fibres having a first alignment and secondary fibres on a different alignment, which serve to space the primary fibres from one another may be provided. One or more filling elements of cotton wool or like material may be provided.

One or more filling elements with fibres of two or more different cross sections may be provided. The fibres of different cross sections may be linear and/or non-linear.

One or more filling elements with fibres provided in a first direction may be provided, with one or more restraining fibres or material. The restraining fibres and/or material may surround and/or enclose and/or be wrapped around and/or contact a plurality of fibres. The restraining fibre or material may be provided as a band. The restraining fibres of material may be provided at the ends of the filling elements and/or at intermediate locations thereon.

One or more filling elements may be provided with peripheral fibres or material provided around the filling element. The peripheral fibres or material may be wrapped around the filling elements in a spiral manner and/or criss-cross manner. The fibres or material may be provided in an anti-clockwise and/or clockwise manner. A fishnet of fibres may be provided around one or more filling elements.

One or more filling elements may be provided with pieces provided therein. The pieces may be intermixed with one or more fibres. The pieces may be spheres, beads, blocks or the like. The pieces may be integral with the fibres and/or connected thereto and/or free to move relative to the fibres. Preferably fibres are wrapped and/or extend around at least part of the periphery of the beads, ideally in a variety of directions. The pieces may be linked together by a fibre or filament, particularly in the case of the series of spheres. The spheres may be surrounded by a mass of braided fibres. The masses of braided fibres may be linked by one or more fibres or filaments. Preferably the masses of fibres surround the spheres.

A single layer of filling elements may be provided within the inner component. Multiple layers of filling elements may be provided within the inner component. One or more intermingled filling elements may be provided within the inner component. The filling elements may be of linear configuration and/or curved and/or wavy. One or more spiral filling elements may be provided. One or more filling elements of substantially circular cross-section may be provided. One or more filling elements with one or more flat surfaces may be provided.

The pores and/or voids and/or apertures and/or gaps occurring in the filling elements and/or there between may be due to the manner of manufacture of the material from which it is formed or may be supplemented with further pores and/or voids and/or apertures or gaps. The supplementation may be provided by degradation and/or absorption of one or more materials forming the filling elements.

The one or more filling elements may be configured and/or formed of one or more materials intended to promote tissue growth, particularly tissue ingrowth through one or more filling elements and/or between the inner component and one or more filling elements and/or between two or more of filling elements. Tissue growth may be promoted by the material type, for instance polyester, included in one or more filling elements. Tissue growth may be promoted by the configuration, particularly the size and/or number of pores and/or gaps and/or apertures in one or more filling elements.

One or more materials used in one or more of the filling elements may be bio-absorbable. The bio-absorbable material may be used to decrease the amount of one or more filling elements present and/or positions at which one or more filling elements is present and/or density at which one or more filling elements is present overtime. The bio-absorbable material may restrain one or more of the filling elements, or a part thereof, in a first state, the bio-absorption of the material allowing one or more filling elements, or a part thereof, to assume a second state. The second state may provide a greater internal volume for one or more filling elements and/or greater porosity for one or more filling elements and/or reduction in mass of one or more filling elements and/or provide more space for tissue ingrowth.

Bio-absorbable material may be incorporated in one or more filling elements by providing areas of bio-absorbable material and/or some fibres of bio-absorbable material. One or more of the one or more filling elements may be entirely bio-absorbable or only partially. Different materials having different rates of bio-absorption may be used for different areas and/or different fibres within one or more filling elements. Slow, moderate and fast bio-absorption materials may be used.

Preferably the core provides equivalent properties and/or behaviour to the nucleus pulposis of a natural disc, for instance during compression and/or distraction and/or horizontal gliding and/or axial rotation and/or flexion and/or extension.

Preferably the position of the core filling is maintained by a spacing component. The spacing component may be a continuation of, and is ideally integral with, the inner component and/or outer component and/or additional elements. The spacing component is preferably a continuation of one or more of the side walls of the inner component and/or the outer component. Preferably the spacing component is only provided on the anterior side of the core. The spacing component may be formed of folded material. The spacing component may be formed of rolled material. The spacing component may be formed of a pad of material.

Preferably the spacing material is formed by a continuation of the outer component extending across the anterior side of the core, preferably on the outside of the core and/or inside the outer component. The continuation may be doubled back on itself once, twice or more. A further continuation of the outer component may extend across the anterior side of the core, preferably on the outside of the core and/or inside the outer component from the other side of the outer component and/or from the other side relative to the core to the continuation. The further continuation may be doubled back on itself once, twice or more. The continuation and further continuation may have one or more parts provided between one or more parts of the other.

The inner component may be an inner jacket. The inner component may be of fabric.

The fabric may be formed by flat or circular weaving, knitting, braiding, embroidery or combinations thereof.

The fabric may be formed using one or more of polyester, polypropylene, polyethylene, carbon fibre, glass fibre, glass, polyaramide, metal, copolymers, polylactic acid, polyglycolic acid, biodegradable materials, silk, cellulose, silk worm silk, spider silk or polycaprolactone.

Preferably the inner component is separate from the core filling element(s). Preferably the inner component is separate from the outer component. Relative movement may be facilitated between the inner and outer components. Relative movement between the inner component and core may be allowed. Preferably movement between the inner and outer components is greater than between the inner component and core filling. Preferably movement between the inner and outer components is facilitated in preference to movement between the inner component and core. Preferably any movement, particularly sliding movement, within the disc is greater between the outer component and inner component than between the inner component and core filling element(s).

The inner component may entirely surround and/or encapsulate the core filling element(s). One or more apertures or gaps are preferred in the inner component, ideally to provide fluid communication through the inner component. Preferably a large number of apertures or gaps are provided the material from which the inner component is formed, for instance a woven fabric. The apertures or gaps occurring in the inner component due to the manner of manufacture of the material from which it is formed may be supplemented with further apertures or gaps. The supplementation may be provided by degradation and/or absorption of one or more materials forming the inner component.

The inner component may be configured and/or formed of one or more materials intended to promote tissue growth, particularly tissue ingrowth between the inner component and the core and/or through the inner component.

One or more materials used in the inner component may be bio-absorbable and/or soluble and/or degradable, particularly with the spine. The bio-absorbable material may be used to decrease the amount of inner component present and/or positions at which the inner component is present and/or density at which the inner component is present overtime. Areas of bio-absorbable material may be provided. Bio-absorbable fibres may be used to form the inner component. The inner component may be entirely bio-absorbable or only partially. Different materials having different rates of bio-absorption may be used. The may be mixed together in the inner component and/or may be used for particular areas thereof and/or in a particular sequence within the inner component. Slow, moderate and fast bio-absorption materials may be used. Preferably bio-absorption of the inner component is used to provide space for tissue ingrowth.

Preferably the inner component provides a smooth inner surface which potentially contacts the filling elements comprising the core, or parts thereof. Preferably uniform contact between the inner surface of the inner component and the core filling element(s) is provided. Preferably the fibres forming the inner surface of the inner component are evenly positioned with respect to one another. Preferably any abrasion of the core filling by the inner component is distributed rather than localised. The inner component preferably provides a smooth inner fabric surface, and ideally woven fibrous surface. A densely packed material may be used for the inner surface, ideally to provide the uniform contact surface with the core. The inner surface of the inner component may be of a different material and/or different configuration to the inside and/or outer surface of the inner component.

The inner component may be formed from a substantially planar element. The inner component may be so formed by folding and/or stitching and/or interdigitating one or more parts thereof. In particular, a top wall of the inner component may be connected to a side wall and hence to a bottom wall. One or more further side walls may be connected to the top wall and/or side wall and/or bottom wall. A series of side walls may be provided by an elongate part of the element. Folds or future folds may define one side wall relative to an adjacent side wall or walls.

In a preferred form, the inner component is formed from an element including a side wall connected on one edge to a top wall and connected on an opposing edge to a bottom wall. The respective edges of the side wall are preferably parallel. It is preferred that the side wall will form the side wall at either the anterior, or more preferably, posterior side. Preferably the side wall is connected on one side edge to one or more other side walls, ideally one. Preferably the side wall is connected on the other side edge to one or more other walls, ideally 4 where the desired is hexagonal when filled and when the desired shape is octagonal when filled. The top and bottom edges of the side walls may be parallel or non-parallel depending upon the locations relative to the top and bottom walls they are to occupy. Preferably all the boundaries between side walls in the strip are parallel to one another.

Preferably the side wall(s), top wall and bottom wall are joined together by stitching and/or other attachment techniques.

One or more of the side walls of the inner component may be reinforced and/or of multiple thickness.

On one or more, preferably all, sides, the inner component may be formed of a plurality of inner components. Such a plurality of inner components may be provided in a spiral form or concentric form. Such a plurality of inner components may be integrally formed or may be separately formed. Preferably the plurality of inner components differ from one another in terms of the material from which they are formed and/or the way in which they are formed and/or the properties they provide.

The reinforcement or multiple thickness may be provided by an additional element provided outside of the side wall. The additional element for a side wall may be provided by wrapping one or more additional elements around the side walls. Preferably additional elements are provided for each side wall. Preferably the additional elements are provided by a continuous band extending around the side of the inner component. Preferably the additional elements are configured to substantially match the dimensions of the side wall they contact.

Additional elements may be provided circumferentially around the filling element(s) and/or inside the outer component. One or more layers of such additional elements may be provided. The one or more layers of additional elements may be free to move relative to one another and/or the core and/or the outer component.

In a preferred form, the additional elements are provided as a continuation of the element providing one or more of the side walls. Preferably the continuation provides 6 or 8 additional elements on the end of the 4 or 6 side walls it already provides.

The additional elements may be joined to the side walls and/or other parts of the inner component by stitching and/or other attachment techniques.

The side walls and/or additional elements may act as an annulus for the disc prosthesis. The side walls and/or additional elements may resist sideways expansion of the core, particularly when under compressive load. The side walls and/or additional elements may provide equivalent properties and/or behaviour to the annulus of a natural disc, for instance during compression and/or distraction and/or horizontal gliding and/or axial rotation and/or flexion and/or extension.

Preferably the inner component the core is filled with filling elements until the core fits snugly within.

The outer component may be an outer jacket. The outer component may be of fabric.

The fabric may be formed by flat or circular weaving, knitting, braiding, embroidery or combinations thereof.

The fabric may be formed using one or more of polyester, polypropylene, polyethylene, carbon fibre, glass fibre, glass, polyaramide, metal, copolymers, polylactic acid, polyglycolic acid, biodegradable materials, silk, cellulose, silk worm silk, spider silk or polycaprolactone.

The outer component may entirely surround the inner component and/or encapsulate the inner component. One or more apertures or gaps are preferred in the outer component, ideally to provide fluid communication through the outer component. Preferably a large number of apertures or gaps are provided the material from which the outer component is formed, for instance a woven fabric. The apertures or gaps occurring in the outer component due to the manner of manufacture of the material from which it is formed may be supplemented with further apertures or gaps. The supplementation may be provided by degradation and/or absorption of one or more materials forming the outer component.

The outer component may be configured and/or formed of one or more materials intended to promote tissue growth, particularly tissue ingrowth through the outer component and/or between the inner component and the core and/or through the inner component, and/or within the core filling elements.

One or more materials used in the outer component may be bio-absorbable and/or soluble and/or degradable, particularly with the spine. The bio-absorbable material may be used to decrease the amount of outer component present and/or positions at which the outer component is present and/or density at which the outer component is present overtime. Areas of bio-absorbable material may be provided. Bio-absorbable fibres may be used to form the outer component. The outer component may be entirely bio-absorbable or only partially. Different materials having different rates of bio-absorption may be used. The may be mixed together in the outer component and/or may be used for particular areas thereof and/or in a particular sequence within the outer component. Slow, moderate and fast bio-absorption materials may be used. Preferably bio-absorption of the outer component is used to provide space for tissue ingrowth.

Preferably the outer component provides a resilient and/or strong containment for the inner component and/or core. Preferably the outer component provides for the anchoring of the prosthesis to the spine.

The outer component may be formed from a substantially planar element. The outer component may be so formed by folding and/or stitching and/or interdigitating one or more parts thereof. In particular, a top wall of the outer component may be connected to a side wall and hence to a bottom wall. One or more further side walls may be connected to the top wall and/or side wall and/or bottom wall. A series of side walls may be provided by an elongate part of the element. Folds or future folds may define one side wall relative to an adjacent side wall or walls.

In a preferred form, the outer component is formed from an element including a side wall connected on one edge to a top wall and connected on an opposing edge to a bottom Wall. The respective edges of the side wall are preferably parallel. It is preferred that the side wall will form the side wall at either the anterior, or more preferably, posterior side. Preferably the side wall is connected on one side edge to one or more other side walls, ideally two. Preferably the side wall is connected on the other side edge to one or more other walls, ideally 2 in the case where the desired core shape after filling is octagonal. A further side wall is preferably connected to the opposite edge of the top wall or bottom wall to the edge to which the side wall linking the top wall and bottom wall is provided. The top and bottom edges of the side walls may be parallel or non-parallel depending upon the locations relative to the top and bottom walls they are to occupy. Preferably all the boundaries between side walls in the strip are parallel to one another.

The inner and/or outer component may be provided with one or more flanges.

In one embodiment, the inner and/or outer component may be provided with a single flange. The flange may be folded across an opening in the inner and/or outer component, for instance to close an opening through which the core filling is inserted or can be accessed. The flange may be attached to the spine, for instance by one or more fixings. The flange may be attached to a vertebra below the prosthesis, relative to the spine of a standing person. The flange may be attached to a vertebra above the prosthesis, relative to the spine of a standing person.

Preferably the inner and/or outer component is provided with at least one flange on one part thereof and at least one other flange on another, preferably opposing, part thereof. Preferably at least one flange which is interdigitated with another, in use, is provided. Preferably one or more edges of the top wall and/or one or more edges of the bottom wall are provided with flanges. Preferably a flange has a length greater than the height of the side walls and/or greater then height of the disc space in which the prosthesis is to be used. The flanges, particularly towards their ends may provide anchor locations for attaching the outer component to one or more vertebrae. Preferably one flange is provided with more anchor locations than another flange, ideally the more anchor locations are provided on the flange for attachment to the inferior and/or lower vertebra. Preferably the one flange is provided with one more anchor locations than the another flange, ideally the more anchor locations are provided on the flange for attachment to the inferior and/or lower vertebra. Preferably the one flange is provided with one anchor location, ideally the more anchor locations are provided on the flange for attachment to the superior and/or upper vertebra. Preferably the another flange is provided with two anchor locations, ideally the more anchor locations are provided on the flange for attachment to the inferior and/or lower vertebra. The anchor locations may be holes, preferably through the flange, and/or fixing receiving locations.

The flanges may have a width less than the width of a side wall. Preferably a first flange has a minimum width less than the minimum width of a second flange, ideally with the one flange having a minimum width less than the minimum width of the another flange. Preferably a first flange has a maximum width less than the maximum width of a second flange, ideally with the one flange having a maximum width less than the maximum width of the another flange. The width of a flange may be considered as the distance from one edge of the flange to another edge in a direction parallel to the disc space and/or perpendicular to the axis of the spinal column and/or across the face of a vertebra, for instance the anterior face. Preferably the first and second flanges, ideally the one flange and the another flange, are of the same length. The length may be considered perpendicular to the width and/or along the axis of the spinal column. Preferably the one flange passes through a hole in the another flange, ideally so as to interdigitated the two flanges.

Preferably a first flange, ideally the one flange, increases in width towards the end of the flange. The first flange, preferably the one flange may taper outward from a reduced neck portion to a wider portion including the anchor location. The wider portion may have a rounded end edge, for instance an edge which has a profile concentric with the fixing. The first flange, ideally the one flange, may be in the form of a finger. Preferably a second flange, ideally the another flange, increases in width towards the end of the flange. The second flange, preferably the another flange may taper outward from a reduced neck portion to a wider portion including the anchor locations. The portion including the anchor locations, particularly a wider portion, may include, at least for a part of the edge, a rounded end edge around each anchor location. The end edge may, in one or more parts, be concentric with a fixing. The portion including the anchor locations, particularly a wider portion, may include a recess in the end edge. The recess may be provided by a part of the flange which is shorted than other parts of the flange, particularly the parts providing the anchor locations. The recess may be provided between the anchor locations and/or part of the flange providing the anchor locations. The recess may be adapted to receive at least a part of the other flange of another disc prosthesis.

The first flange, ideally the one flange, may form a part of the anterior surface profile of the disc prosthesis. Preferably it provides the stem of a Y-shaped profile. Preferably the second flange, ideally the another flange, forms part of the anterior surface profile of the disc prosthesis. Preferably it provides the forks of a Y-shaped profile. Preferably at least a part of the anterior profile of one disc prosthesis, particularly a part of the stem of a Y-shaped profile, may be received between parts of the anterior profile of another disc prosthesis, particularly between the forks of a Y-shaped profile. The at least part of the anterior profile may be so received without any overlap in the material of the one disc prosthesis with the material of the another disc prosthesis.

In a preferred form, a flange is provided on an edge of the top wall which opposes, ideally when considered in the assembled position, an edge of the bottom wall provided with a flange. One of the flanges may be provided with a through aperture. One of the flanges may be provided with a reduced width and/or neck part. Preferably one of the flanges is interdigitated with the other by passing it though the hole. The flange from the top wall is preferably anchored to the bottom vertebrae and the flange from the bottom wall is preferably anchored to the top vertebrae, relative to the disc space being treated, in such a case. One or more pairs of flanges of this type may be provided. The flanges in a pair of flanges may be joined to one another, for instance by a web. The pair of flanges and web may define, at least in part, the boundaries of an aperture.

Preferably the side wall(s), top wall and bottom wall are joined together by stitching and/or other attachment techniques.

The side walls of the outer component may act as an annulus for the disc prosthesis. The side walls of the outer component may resist sideways expansion of the core, particularly when under compressive load. The side walls of the outer component may provide equivalent properties and/or behaviour to the annulus of a natural disc, for instance during compression and/or distraction and/or horizontal gliding and/or axial rotation and/or flexion and/or extension.

Preferably the inner component is provided snugly within the outer component. Preferably the top wall and/or bottom wall and/or one or more side walls of the outer component are dimensioned to contact the inner component.

Preferably the prosthetic disc is anchored to the spine away from the anterior side. Preferably the anchor positions are provided to either side of the anterior of the spine. One or more anchor positions may be used, preferably at least two are used on the vertebrae above and two on the vertebrae below the disc being replaced.

Preferably the prosthetic disc is anchored to the spine using one or more anchor locations provided thereon. Preferably one or more anchor locations are provided by a flange or flanges provided by the inner and/or outer component. Preferably a flange has a length greater than the height of the side walls and/or greater then height of the disc space in which the prosthesis is to be used. The flanges may provide the anchor locations towards their ends. The flanges may have a width less than the width of a side wall.

In a preferred form, a flange is provided on the inner and/or outer component in opposition to another flange provided on another part of the inner and/or outer component. One of the flanges may be provided with a through aperture. One of the flanges may be provided with a reduced width and/or neck part. Preferably one of the flanges is interdigitated with the other by passing it though the hole. The flange from the top wall is preferably anchored to the bottom vertebrae and the flange from the bottom wall is preferably anchored to the top vertebrae, relative to the disc space being treated, in such a case. One or more pairs of flanges of this type may be provided.

The inner and/or outer component may be fastened at the anchor positions to one or more adjacent vertebra, for instance using fasteners. The fasteners may be one or more of bone screws, staples, sutures, nails or the like.

The disc prosthesis may include absorbable, for instance bio-absorbable, material between the anchor position or positions of the prosthesis and the outer component of the prosthesis. The disc prosthesis may include absorbable, for instance bio-absorbable, material between a part of the flange or flanges of the prosthesis and the outer component of the prosthesis.

The anchor position(s) and/or at least a part of the flange(s) may be joined to the disc prosthesis, particularly the outer component thereof, by an absorbable zone. The absorbable zone may be formed entirely of absorbable material. The absorbable material may be made of fibres. The absorbable zone may provide the only joint with the disc prosthesis, particularly the outer component thereof. The absorbable zone may make the anchor position(s) and/or at least a part of the flange(s) detachable from the disc prosthesis, particularly the outer component thereof.

The anchor position(s) and/or more particularly at least a part of the flange(s) may be formed from at least two different materials. At least one absorbable material is preferably provided. At least one non-absorbable material is preferably provided. Preferably at least one of the materials is used to provide the load bearing function, preferably the load bearing fibres. Preferably the load bearing material is made of an absorbable material, particularly absorbable fibres. Preferably the at least one non-absorbable material defines the overall shape of the flange(s) and/or maintain the interdigitation of flanges and/or is subjected to level of tension, particularly after absorption of the absorbable material. The absorbable material may surround the non-absorbable material.

The anchor position(s) and/or at least a part of the flange(s) may be joined to the disc prosthesis, particularly the outer component, by a plurality of different material, particularly fibre, configurations and/or types. A material having a non-linear configuration, particularly in terms of the fibres forming it may be provided. The non-linear material and/or fibres may be curved and/or spiraled and/or serpentine and/or zigzag in configuration. The non-linear material and/or fibres may have a first form and a second form. In the second form, the length of the material and/or fibres being greater in the second form and/or the material and/or fibres may be more linear. Preferably the non-linear material and/or fibres are not load bearing at the first time and/or at implantation and/or in the first form. The non-linear material and/or fibres may be maintained in the first form by a further material and/or further fibres. The further material and/or fibres may be absorbable. Preferably the further material and/or further fibres are load bearing at the first time and/or at implantations and/or in their first form. Preferably the further material and/or fibres are present in their first form and absent, preferably due to absorption, in their second form. The non-linear material and further material may be separate from one another. The further material may surround the non-linear material, for instance as a sleeve. The further material may be mixed or intermingled with the non-linear material. The further material may isolate the non-linear material from the load in the first form. The further material may be attached to the non-linear material in the first form. The attachment may be through adhesion to and/or winding round and/or stitching to the further material. The further material may act as a bridging material between parts of the non-linear material.

The absorbable material may be provided in one or more forms. A plurality of forms may be provided. The plurality of forms may provide for different rates of absorption. The different forms may different in terms of one or more of their material and/or diameters and/or dimensions and/or densities and/or bulk densities. The absorbable materials and/or non-absorbable materials may be provided in one or more in-growth controlling forms. Different in-growth controlling forms may be used to give different extents of tissue ingrowth with time. Different in-growth controlling forms may be used to give different in-growth extents for different parts of the prosthesis, and particularly within different parts of the flanges. The different extents may be between zero and the maximum possible.

The anchor position(s) and/or the flange(s) may be provided with suture receiving sections. The suture receiving sections may be provided on all flanges and preferably define the anchor positions. The suture receiving sections may include one or more suture bearing parts. The suture bearing parts may be reinforced parts, for instance one or more reinforced bands. One of more of the suture receiving parts may extend across the flange and/or perpendicular to the direction of load and/or tension. One or more of the suture receiving parts may extend across the flanges between fibres, particularly load bearing fibres, on one side of the flange and fibres, particularly load bearing fibres, on the other side of the flange. A series of suture receiving sections are preferably provided, preferably spaced along the length of the flanges. Between the suture bearing parts, one or more openings may be provided. Preferably one or more of the openings are spanned by one of more fibres, and ideally by a mesh. Preferably a suture is passed through the opening, round the suture bearing part and through an opening on the other side of the suture bearing part. Preferably multiple loops of the suture are provided. Preferably a plurality of anchor positions are provided along the length of the flange(s). Preferably a plurality of suture receiving sections and/or suture bearing parts are provided along the length of the flange.

The first aspect of the invention may include any of the features, options or possibilities set out elsewhere in this document.

According to a second aspect of the invention we provide a kit for use in providing a disc prosthesis, the kit including a series of different sized prostheses, one or more of the prostheses including a core, the core being filling elements positioned within an inner component, the inner component being provided within an outer component.

Preferably the kit includes different sized prostheses for different sized patients and/or different sized prostheses sized for different discs of the spine and particularly the lumber region thereof.

The second aspect of the invention may include any of the features, options or possibilities set out elsewhere in this document.

According to a third aspect of the invention we provide a surgical technique for providing a disc prosthesis, the technique including, removing at least part of the natural disc in a spine and inserting a disc prosthesis in the spine, the disc prosthesis comprising a core formed of one or more filing elements. The core being filling elements provided within an inner component, the inner component being provided within an outer component.

The technique may be performed via an anterior approach, a posterior approach, a lateral approach, an antero-lateral approach, and/or a postero-lateral approach.

The method may include forming the core in-situ. For instance, multiple filling elements may be used to form the core. The method may be a minimally invasive surgical technique, particularly where the core is formed in the inner component in-situ. The inner component may be inserted and then filled with the core. The outer component may be inserted then have the inner component provided within it, potentially then being filled with core.

The technique may use a pre-assembled prosthesis. Preferably the outer component is inserted into the space and the inner component and core are then inserted. The inner component and core may be provided pre-assembled with the core filing element(s) already filled within the inner component.

Preferably the level of tension and/or load between the anchor position or positions of the disc prosthesis and the outer component of the disc prosthesis vary between a first time and a second time. The first time may be the time of implantation, for instance 1 hour after implantation, or perhaps 1 day after implantation. The second time may be a time after implantation, for instance at least 30 days, preferably at least 60 days, more preferably at least 100 days and potentially even at least 300 days, after implantation. Preferably the level of tension and/or load is lower at the second time than at the first time. Preferably the level of tension and/or load is lower after biological in-growth has occurred. The ingrowth may be into the outer component and/or inner component and/or flanges. Preferably the range of extension of the spine at the first time is less than the range of extension at the second time. Preferably the transition between the level of load and/or level of tension and/or range of extension at the first time and at the second time is phased or gradual. The transition may occur evenly through out the time between the first time and the second time, but preferably occurs during a time period starting after the first time. The transition may continue after the second time to a still lower level of tension and/or load and/or to a still higher range of extension.

The method may include using a disc prosthesis provided with at least one flange on one part thereof and at least one other flange on another, preferably opposing, part thereof. Preferably the method includes at least one flange being interdigitated with another flange, preferably by passing the one flange through a hole in the another flange. The method may include introducing one or more fixings to anchor locations, preferably provided towards the ends of the flange(s). Preferably the method includes providing one flange with more fixings than another flange, ideally the more fixings are provided on the flange for attachment to the inferior and/or lower vertebra. Preferably the method includes provided one flange with one more fixing than the another flange, ideally the more fixings are provided on the flange for attachment to the inferior and/or lower vertebra. Preferably the method includes providing the one flange with one fixing, ideally the one fixing is provided on the flange for attachment to the superior and/or upper vertebra, and providing the another flange with two fixings, ideally the two fixings are provided on the flange for attachment to the inferior and/or lower vertebra.

The method may include using a flange provided with a recess, particularly in the end thereof. The end may be that part of the flange furthest from the core. The method may include providing fixings through the flange to either side of the recess. The method may include providing a further disc prosthesis, preferably of the same type, for an adjacent disc space to that the disc prosthesis is provided in. The method may include fixing a flange of the disc prosthesis and a flange of the further disc prosthesis to the same vertebra. The method may include a providing at least a part of one disc prosthesis between at least a part of another disc prosthesis. The part may be provided within the recess. The part may be provided within a recess provided between the anchor locations and/or part of the flange providing the anchor locations and/or the fixings.

The method may include the use of a first flange, ideally the one flange, to form a part of the anterior surface profile of the disc prosthesis. Preferably the method includes the provision as a part of the profile of the stem of a Y-shaped profile. Preferably the method includes the use of a second flange, ideally the another flange, to form part of the anterior surface profile of the disc prosthesis. Preferably the method includes the provision as a part of the profile of the forks of a Y-shaped profile. Preferably at least a part of the anterior profile of one disc prosthesis, particularly a part of the stem of a Y-shaped profile, is provided between parts of the anterior profile of another disc prosthesis, particularly between the forks of a Y-shaped profile, as a part of the method. The method preferably includes the at least part of the anterior profile being so provided without any overlap in the material of the one disc prosthesis with the material of the another disc prosthesis.

The third aspect of the invention may include any of the features, options or possibilities set out elsewhere in this document.

According to a fourth aspect of the invention we provide a disc prosthesis, the disc prosthesis including an outer component, the outer component being provided with at least one flange on one part thereof and at least one other flange on another part thereof.

Preferably at least one flange which is interdigitated with another, in use, is provided. Preferably one or more edges of the top wall and/or one or more edges of the bottom wall are provided with flanges. Preferably a flange has a length greater than the height of the side walls and/or greater than height of the disc space in which the prosthesis is to be used. The flanges, particularly towards their ends may provide anchor locations for attaching the outer component to one or more vertebrae. Preferably one flange is provided with more anchor locations than another flange, ideally the more anchor locations are provided on the flange for attachment to the inferior and/or lower vertebra. Preferably the one flange is provided with one more anchor locations than the another flange, ideally the more anchor locations are provided on the flange for attachment to the inferior and/or lower vertebra. Preferably the one flange is provided with one anchor location, ideally the one anchor location is provided on the flange for attachment to the superior and/or upper vertebra and the another flange is provided with two anchor locations, ideally the two anchor locations are provided on the flange for attachment to the inferior and/or lower vertebra. The anchor locations may be holes, preferably through the flange, and/or fixing receiving locations.

The flanges may have a width less than the width of a side wall. Preferably a first flange has a minimum width less than the minimum width of a second flange, ideally with the one flange having a minimum width less than the minimum width of the another flange. Preferably a first flange has a maximum width less than the maximum width of a second flange, ideally with the one flange having a maximum width less than the maximum width of the another flange. The width of a flange may be considered as the distance from one edge of the flange to another edge in a direction parallel to the disc space and/or perpendicular to the axis of the spinal column and/or across the face of a vertebra, for instance the anterior face. Preferably the first and second flanges, ideally the one flange and the another flange, are of the same length. The length may be considered perpendicular to the width and/or along the axis of the spinal column. Preferably the one flange passes through a hole in the another flange, ideally so as to interdigitated the two flanges.

Preferably a first flange, ideally the one flange, increases in width towards the end of the flange. The first flange, preferably the one flange may taper outward from a reduced neck portion to a wider portion including the anchor location. The wider portion may have a rounded end edge, for instance an edge which has a profile concentric with the fixing. The first flange, ideally the one flange, may be in the form of a finger. Preferably a second flange, ideally the another flange, increases in width towards the end of the flange. The second flange, preferably the another flange may taper outward from a reduced neck portion to a wider portion including the anchor locations. The portion including the anchor locations, particularly a wider portion, may include, at least for a part of the edge, a rounded end edge around each anchor location. The end edge may, in one or more parts, be concentric with a fixing. The portion including the anchor locations, particularly a wider portion, may include a recess in the end edge. The recess may be provided by a part of the flange which is shorted than other parts of the flange, particularly the parts providing the anchor locations. The recess may be provided between the anchor locations and/or part of the flange providing the anchor locations. The recess may be adapted to receive at least a part of the other flange of another disc prosthesis.

The first flange, ideally the one flange, may form a part of the anterior surface profile of the disc prosthesis. Preferably it provides the stem of a Y-shaped profile. Preferably the second flange, ideally the another flange, forms part of the anterior surface profile of the disc prosthesis. Preferably it provides the forks of a Y-shaped profile. Preferably at least a part of the anterior profile of one disc prosthesis, particularly a part of the stem of a Y-shaped profile, may be received between parts of the anterior profile of another disc prosthesis, particularly between the forks of a Y-shaped profile. The at least part of the anterior profile may be so received without any overlap in the material of the one disc prosthesis with the material of the another disc prosthesis.

The fourth aspect of the invention may include any of the features, options or possibilities set out elsewhere in this document.

According to a fifth aspect of the invention we provide a surgical technique for providing a disc prosthesis, the technique including, removing at least part of the natural disc in a spine and inserting a disc prosthesis in the spine, the disc prosthesis including an outer component, the outer component being provided with at least one flange on one part thereof and at least one other flange on another part thereof.

The method may include using a disc prosthesis provided with at least one flange on one part thereof and at least one other flange on another, preferably opposing, part thereof. Preferably the method includes at least one flange being interdigitated with another flange, preferably by passing the one flange through a hole in the another flange. The method may include introducing one or more fixings to anchor locations, preferably provided towards the ends of the flange(s). Preferably the method includes providing one flange with more fixings than another flange, ideally the more fixings are provided on the flange for attachment to the inferior and/or lower vertebra. Preferably the method includes provided one flange with one more fixing than the another flange, ideally the more fixings provided on the flange for attachment to the inferior and/or lower vertebra. Preferably the method includes providing the one flange with one fixing, ideally the one fixing is provided on the flange for attachment to the inferior and/or lower vertebra and providing the another flange with two fixings, ideally the two fixings are provided on the flange for attachment to the inferior and/or lower vertebra.

The method may include using a flange provided with a recess, particularly in the end thereof. The end may be that part of the flange furthest from the core. The method may include providing fixings through the flange to either side of the recess. The method may include providing a further disc prosthesis, preferably of the same type, for an adjacent disc space to that the disc prosthesis is provided in. The method may include fixing a flange of the disc prosthesis and a flange of the further disc prosthesis to the same vertebra. The method may include a providing at least a part of one disc prosthesis between at least a part of another disc prosthesis. The part may be provided within the recess. The part may be provided within a recess provided between the anchor locations and/or part of the flange providing the anchor locations and/or the fixings.

The method may include the use of a first flange, ideally the one flange, to form a part of the anterior surface profile of the disc prosthesis. Preferably the method includes the provision as a part of the profile of the stem of a Y-shaped profile. Preferably the method includes the use of a second flange, ideally the another flange, to form part of the anterior surface profile of the disc prosthesis. Preferably the method includes the provision as a part of the profile of the forks of a Y-shaped profile. Preferably at least a part of the anterior profile of one disc prosthesis, particularly a part of the stem of a Y-shaped profile, is provided between parts of the anterior profile of another disc prosthesis, particularly between the forks of a Y-shaped profile, as a part of the method. The method preferably includes the at least part of the anterior profile being so provided without any overlap in the material of the one disc prosthesis with the material of the another disc prosthesis.

The fifth aspect of the invention may include any of the features, options or possibilities set out elsewhere in this document.

In a sixth aspect of the invention filling elements may be introduced into (by way of example only) the outer component via a filling instrument. In this instance, the outer component is not provided with an opening in an end, but instead is filled through a pore in the outer component. The filling instrument may have a tip of reduced diameter dimensioned to be pushed into the pore. In doing so, the size of the pore is increased by stretching to be greater than the size of the tip. The filling elements (e.g. one or more filaments, etc. . . . ) can then be injected. The density of the filling element(s) is such that it/they can readily flow under pressure from the injection tool through a stretched opening. However, the density is such that the filling elements cannot readily flow through an outstretched opening, particularly when under the lower pressure levels experienced within the outer component compared with those experienced in the tip. The same principle would apply where the filling includes distinct particles such as beads. The stretched pore is large enough to allow the filling in, but the normal size pores are too small to allow the filling out.

Whilst the inner and/or outer component can be entirely flexible, consistent with its fabric/textile nature, there are benefits in providing a more defined structure or profile to one or both of these components. Thus, as shown by way of example the outer component is provided of fabric, but within the bag a number of stiffening elements are provided. Thus a series of stiffening elements are provided in the form of rings which extend around the periphery of the outer component and so seek to maintain the side wall profile of the outer component. For insertion, the sides of the rings can be squeezed together and so reduce the cross-section of the outer component. Once inside the disc space, the compression can be removed and the rings will push the sides of the outer component outwards to the disc like profile. This assists in ensuring the shape of the implant is correct and assists in providing the space into which the filling elements can be introduced.

The stiffening elements may also be configured to push the top and bottom rings apart in a vertical sense. Again a downward compression can be used to reduce the profile of the outer component, with the removal of that compression allowing the outer component to return to the desired form.

Such arrangements of stiffening elements can be used to close or assist in supporting the closure of the inner and/or outer component. Equally, such stiffening elements can be used to support surfaces of the inner and/or outer component against loads. For instance, the surface of the implant which faces the vertebra above the implant in a standing individual and/or the surface which faces the vertebra below may be provided with stiffening elements which extend across them to resist loads. Stiffening elements down the sides, round the edges and at other positions may also be provided to support the shape of the implant and/or contribute to its functional characteristics. Resistance to load, extension, compression, flexion or the like may be provided in this way, as might resistance to tissue ingrowth pressures.

The characteristics provided by the stiffening elements may be different for different parts of the implant. For instance, some parts may be less resistant to a force than others. Metal wires, metal fibres, stiff plastics wires or fibres and the like could be used for the stiffening elements. In particular shape memory materials, such as nitinol, could be used for the stiffening elements. A wide variety of configurations are possible, including rings, spirals, zig-zags, loops, coils, waves and others.

In an seventh aspect of the present invention, the fibres which are woven together to form the outer component include on their outside a series of projections. The projections are integrally formed with the fibres and are provided at an inclined angle. As such, as the outer component expands during filling and/or moves during insertion/filling/use, the projections act as barbs and dig into the surrounding material of the annulus. In this way, a firm anchorage for the implant is provided all over its surface, including those parts which could not be reached from the small incision used to insert the implant. If sutures or staples are to be used to fix the implant within the annulus, then they can only really be provided at or close to the incision site.

### BRIEF DESCRIPTION OF THE DRAWINGS

Various embodiments of the invention will now be described, by way of example only, and with reference to the accompanying drawings in which:

FIG. 1 shows a perspective view of a disc featuring part of a device according to an embodiment of the present invention;

FIG. 2 shows the view of FIG. 1 with the device near completion;

FIG. 3 shows the dispensing of one embodiment of the filling using one embodiment of an applicator;

FIGS. 4a to 4c show other embodiments of fillings;

FIG. 5 shows a further embodiment of a filing in perspective view;

FIGS. 6a and 6b shows still further embodiments of filings in perspective view;

FIG. 7 shows yet another embodiment of a filling;

FIG. 8 shows an embodiment of the invention including beads;

FIG. 9 shows a further bead incorporating embodiment of the invention;

FIG. 10a to 10c show different stages in the life of a device according to the invention, from initial point of deployment, through an intermediate time to a much later time after deployment;

FIG. 11 is a plan view comparing the profile of a core according to the invention with a natural disc;

FIG. 12 illustrates an inner jacket according to the present invention, prior to assembly;

FIG. 13 illustrates an outer jacket according to the present invention, prior to assembly;

FIG. 14 illustrates an outer jacket according to another embodiment of the present invention, prior to assembly;

FIGS. 15a, 15b and 15c show respectively an assembled disc outer, disc outer in plan view and disc outer in combination with core;

FIG. 16a, 16b and 16c show respectively an assembled disc outer with an inner, annular reinforcement, the disc outer in plan view and the disc outer in plan view with the inner annular reinforcement and core;

FIGS. 17a and 17b show respectively an assembled disc outer with inner reinforcement and core and plan view of the same;

FIG. 18a illustrates a further embodiment of the outerjacket prior to assembly;

FIG. 18b illustrates the embodiment of FIG. 18a in assembled format in a plan view;

FIG. 18c illustrates the embodiment of FIG. 18a in assembled, perspective view;

FIG. 19a illustrates a view of an embodiment of an inner reinforcement, prior to assembly;

FIG. 19b illustrates the outer of FIG. 19a in assembled form, in plan view;

FIG. 19c shows the inner of FIG. 19a in assembled form, and contained within an outer jacket;

FIG. 20 shows a still further embodiment of an outerjacket, prior to assembly;

FIG. 21a shows an embodiment of a disc outer potentially assembled from a disc outer according to FIG. 20;

FIG. 21b shows an assembled disc outer with buttress elements, potentially formed from an outer jacket according to FIG. 20;

FIG. 21c shows an assembled disc outer with buttress elements, potentially formed from an outer jacket according to FIG. 20;

FIG. 21d is a perspective view of an assembled outer jacket including the buttress elements;

FIG. 22a shows another embodiment of an outer jacket, prior to assembly;

FIG. 22b shows the embodiment of FIG. 22a, with certain sections highlighted;

FIG. 23 illustrates an assembled outer jacket according to one form, left hand side, and according to another form, right hand side;

FIG. 24 illustrates the use of two assembled discs, with outer jackets according to the another form of FIG. 23, between adjacent vertebrae;

FIG. 25 illustrates in a closer view the use of two assembled discs, with outer jackets according to the other form of FIG. 23, between adjacent vertebrae;

FIG. 26 shows another embodiment of the invention in perspective view with the filling elements being inserted;

FIG. 27 shows stiffening elements incorporated according to another embodiment of the invention;

FIG. 28 shows an alternative form of stiffening elements; and

FIG. 29 shows a form of anchoring between the implant and surrounding tissue.

### DESCRIPTION OF THE PREFERRED EMBODIMENT

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure. The systems disclosed herein boast a variety of inventive features and components that warrant patent protection, both individually and in combination.

The prior art contains examples of elastomeric discs, with the motion of the elastomer being contained by bonding it to metallic end-plates. In use, this results in high strains at the exterior faces of the disc and this in turn can give rise to tearing and eventually failure of the core.

The previously developed artificial intervertebral disc detailed in U.S. Pat. No. 6,093,205, was developed particularly for the cervical region of the spine. The combination of an elastomeric inner core surrounded by a single embroidered outer textiles jacket has been shown to offer particular benefit in terms of the encapsulation preventing the initiation or propagation of any fissures in the elastomer component of the artificial disc.

To provide an optimised artificial disc for use in the lumbar region of the spine a number of further developments and improvements have been made, including but not necessarily limited to, encasing a core of filling elements within an inner component (or inner jacket), which in turn, may be contained within an outer component (or outer jacket). This advancement allows relative movement between the core and its encasing jacket to be minimized while still allowing a desired level of movement between the implant and the vertebrae overall. The artificial disc may act as a complete disc replacement, or a partial replacement, for instance for the nucleus. Anterior, posterior, or lateral insertion is possible. The further developments and improvements are also useful in the context of other disc prostheses too.

A variety of core designs are possible whilst providing optimal performance.

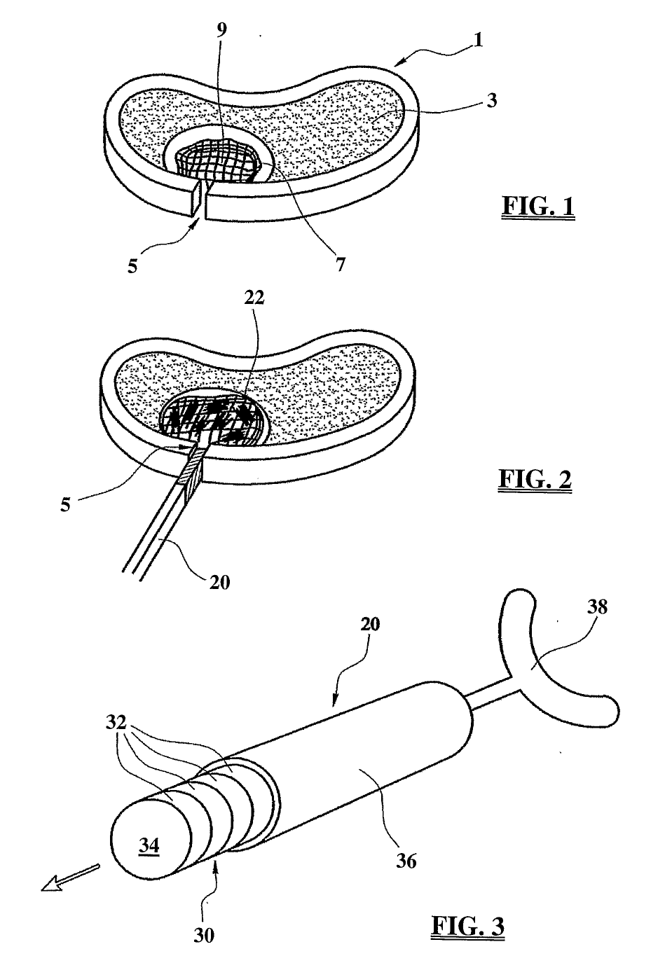

With reference to FIG. 1, an intervertebral disc 1 is shown with part of the nucleus 3 removed through an incision 5. Following removal of the nucleus material, a first part of an implant according to a first embodiment (and shown by way of example only) may be inserted, as pictured in FIG. 1. The first part is a fabric bag 7 with an opening 9. The bag 7 is empty and hence easily reduced to a small size at this stage so as to allow easy insertion through the incision 5. The incision 5 is of the smallest size necessary to remove the nucleus material. This contrasts with prior art systems where the incision 5 needed for the nucleus removal needed to be enlarged to allow enough room to deploy the implant. The opening 9 into the bag is kept close to the incision 5.

The bag 7 is formed in such away as to offer the necessary strength and structural properties to constrain the core (e.g. filling elements described below) it is to receive, but does so whilst being open to the passage of fluid through it, both into and out of its inside. The significance of this will be described in greater detail below. As shown and described herein, bag 7 preferably comprises an inner jacket for encasing an implant core formed of one or more filling elements.

Bag 7 is shown here without concurrent use of an outer jacket for the purposes of clarity only. It will be appreciated however that such an outer jacket is contemplated and preferred. It will also be appreciated that bag 7 may alternatively form an outer jacket and a second bag 7 may form an innerjacket.

In FIG. 2, the next stage of the implants formation is shown. Using an applicator 20, a second part of the implant, the core comprising filling elements 22 is pushed into the bag 7 through the opening 9. The filling elements 22 is/are of relatively small cross-section and so does not necessitate any enlargement of the incision 5 either. A sufficient amount of filling element 22 is introduced into the bag 7 to give it the desired properties, as discussed in more detail below. As can be seen, however, the filling 22 causes the bag 7 to generally assume the profile of the space in the nucleus 3.

The filing elements 22 may be made of one or more materials which encourage tissue growth, such as polyester fibre.

Such a bag can be provided together with (as in used alongside but discrete from), linked to, or as an integral part of the type of device disclosed in applicant's UK patent application no 0406835.9 filed 26 Mar. 2004, the contents of which are incorporated herein by reference with respect to that device.

An implant according to the present invention is suitable when a procedure such as a nucleotomy has been conducted as the disc will have lost material from the nucleus. This may cause a loss in nucleus function and/or a loss in disc height. The implant thus provides a partial artificial disc and so provides treatment in these cases. A full disc replacement can also be performed.

An important part of the present invention is the filling 22 used to form the core and the structure of the bag 7 used to form the inner (and/or outer) jacket component.

In disc/nucleus replacement procedures, the prior art approach has been to provide a non-biological mechanism for mimicking the disc's natural function throughout the life of the device. As far as practically possible the device has been isolated from its biological surrounds. The present invention aims to provide a phased transition from a solution based on a non-biological mechanism to a combination of biological and non-biological mechanisms and potentially even on to a predominantly or even exclusively biological mechanism.

This aim can be achieved by careful design of core filling elements 22 and bag 7 to facilitate rather than resist tissue ingrowth.

When exposed to alien materials which cannot be expelled or broken down, the body's reaction is to try and isolate the material. Tissue thus grows around the material.

In the past, the continuous nature of the implant has meant that the tissue has grown only around the outside of the implant. In the case of inflatable balloons, this is because the outer element which constrains the inflation, by its very nature, also prevents tissue growth inside. Similarly metal devices prevent tissue ingrowth because of the material they are made from. Other implants have used an outer element which is continuous in nature and so only a surface layer of tissue around the very outside may have developed. Either because to the nature of the implant or because of active steps taken, no tissue ingrowth within the implant occurs. In some cases, steps to actively avoid tissue ingrowth have been taken, for instance to prevent the tissue interfering with the operation of the non-biological mechanics of the device.

The present invention takes a fundamentally different approach and actively seeks tissue ingrowth for the implant.

Firstly, the bag 7 is provided in such a way that there are significant openings/gaps between the fibres forming the bags. Fluids can thus readily pass through the bag 7 in either direction. As a result, the outer and/or inner components of the implant facilitate tissue ingrowth through themselves.

Secondly, and with reference to another embodiment, shown by way of example only in FIG. 2, the filling elements 22 (and thus the core) consist of groups of fibres collected together in an unconstrained, unbraided mass. The elongate nature of the fibres suits them to alignment within the applicator 20. Some alignment of the fibres is retained within the bag 7, but generally the result is a core formed of an open mass of fibres.

Such a filling 22 of unconstrained and unbraided polyester filaments or fibres initially occupies a small volume in the nucleus. Following implantation, however, tissue ingrowth into the core filling 22 occurs. The open nature of the mass of fibres and material of the fibres promotes this. With time, the tissue ingrowth tends to surround each fibre individually, as the tissue is able to reach each individually. Thus each individual fibre is alien material to be isolated by surrounding. If densely packed fibres are provided, the tissue growth is again restricted to the outside as the fibres are seen as an integral mass by the tissue. The open fibres of the present invention in effect act as a scaffold. As this growth progresses, it will cause the volume of the filling 22 and hence the bag 7 to swell to fill the available space in the nucleus.

The lack of restriction on the tissue ingrowth and the free access for fluids into and out of the bag 7 and filling elements 22 should mean that the tissue which grows is similar in composition and hence properties to the undisturbed nucleus material that surrounds it.

The swelling of the bag 7 should restore some of the disc height that has been lost as the disc failed.

In theory, during the earlier stages of degenerative disc disease, the idea of refilling the nucleus with scaffolding polyester fibre could act as a permanent treatment. At the very least, it would be expected to improve the patient's condition in the medium term delaying a more serious procedure. In the meantime, all normal treatment options would still be able to be used on the patient.

An applicator 20 is illustrated in more detail in FIG. 3, in conjunction with a different form of core filling elements 30. In this case, rather than being a mass of fibres in an unconstrained form (as in FIG. 2), the filling elements 30 are provided in the form of a number of discrete pads 32 of felt like material 34. Felt and similar materials used the natural interlacing of their fibres to form an open porous structure. This can be supplemented by needling to increase the interlacing and/or openness of the structure if desired.

Applicator 20 consists of a tube 36 which holds the pads 32. Under the control of the surgeon a plunger 38 is advanced in the tube 36 to push the pads 32 out into the bag 7 within the disc. Overtime, the pads 32 expand as tissue grows within and around them. Different applicator cross-sections can be used to deploy different fillings (e.g. fibres, filaments, pads, etc. . . . ).

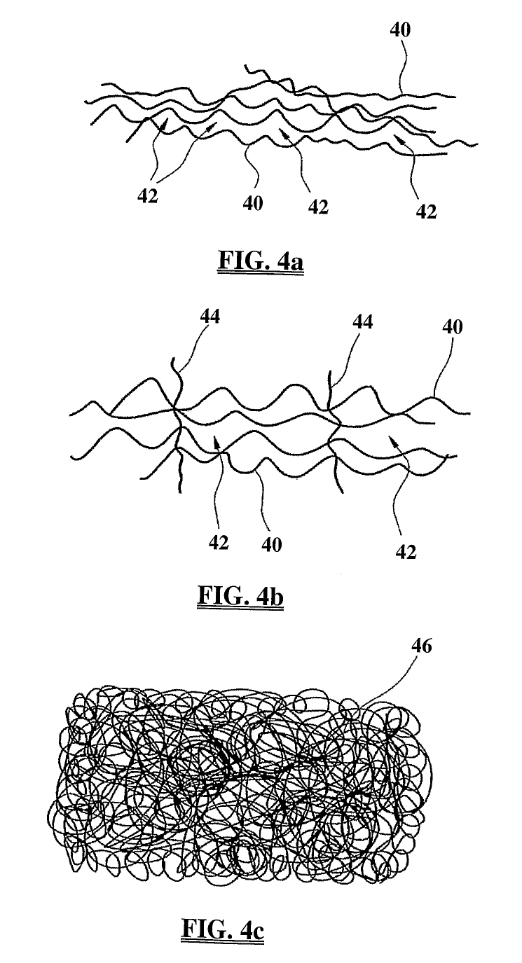

FIGS. 4a, 4b and 4c illustrate a number of alternative forms of filling 22 in unconstrained, unbraided form. FIG. 4a shows a series of generally aligned fibres 40 which are non-linear in nature. The waves built into the fibres 40 serve to space individual fibres 40 from one another. The result is a mass of fibres 40 with substantial voids 42. FIG. 4b shows a modification, in which a series of secondary fibres 44 are provided with a different orientation to the primary fibres 40. The difference in orientation resists pressures which would otherwise cause the voids 42 between the fibres to be reduced. FIG. 4c shows a mass of fibres 46 in a form more closely approaching that of a felt or cotton wool material. A very large number of different orientations are provided and thus serve to maintain the spacing against compression in a wide variety of directions,

The fibre could be provided from staple fibre, potentially subsequently chopped into short lengths. The fibre could be used as supplied, or be modified before or after chopping, potentially to provide braiding or other restraining surround. It is possible to use fibres formed of single filaments and/or filaments twisted together and/or braided together.

FIG. 5 shows a further filling element form in which primary fibres 650 of a large cross-section are mixed with secondary fibres 652 of a smaller cross-section. The differences in cross-section again help to maintain the voids 654 within the filling.

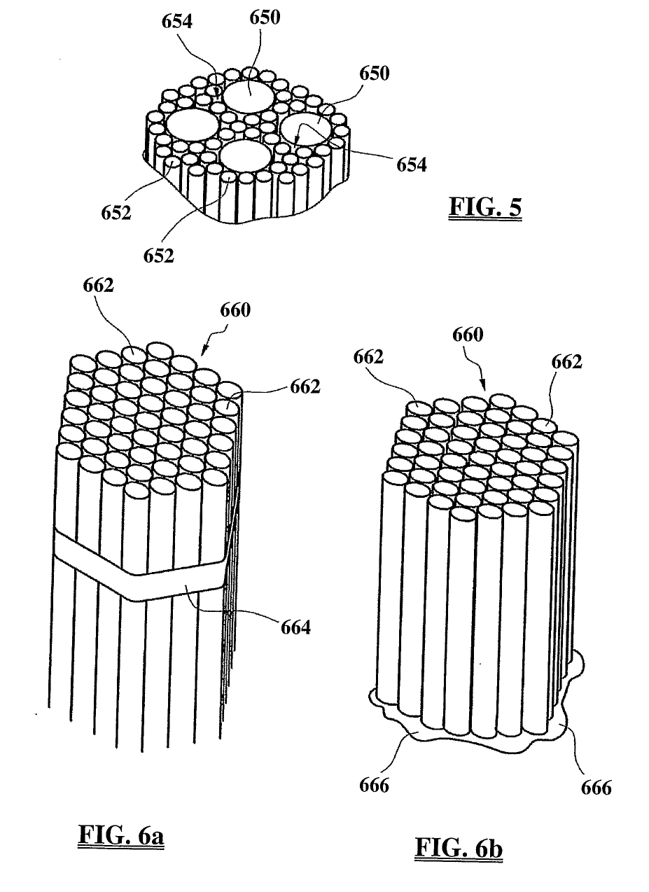

FIGS. 6a and 6b illustrate examples of a more structured filling element 660. In the first case, FIG. 6a, the majority of the fibres 662 are provided along a first alignment. To assist in keeping the alignment of the fibres 662 during and after deployment, a limited number of fibres 664 are wrapped around the fibres 662 to maintain them as bundles. The bundles are still open, however, and have significant voids. In the FIG. 6b form, the fibre bundle is chopped by a hot blade and this melts part of the ends and joins them together upon cooling due to mass 666.

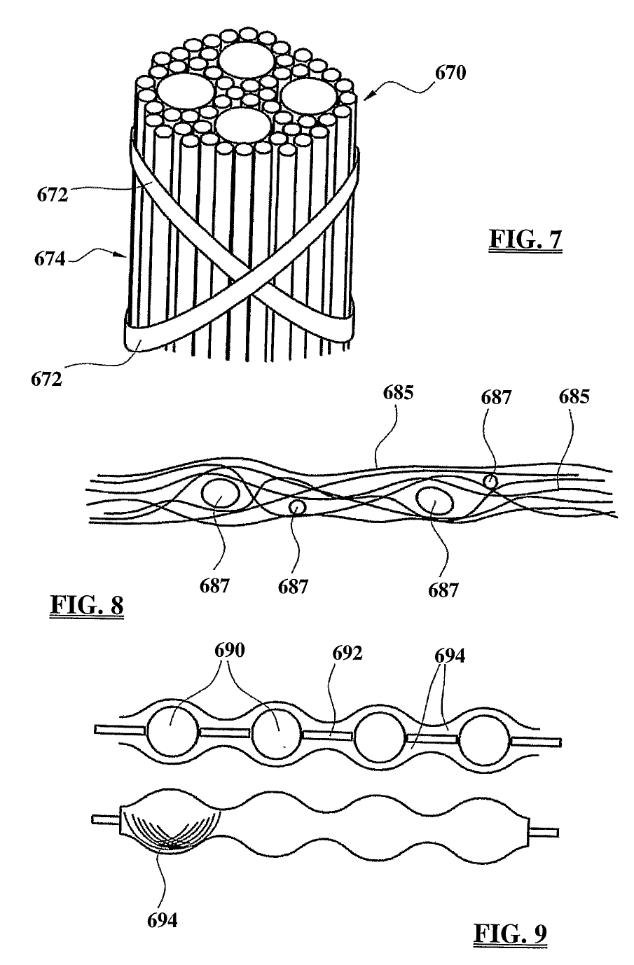

Turning to FIG. 7, a still more structured embodiment of core filling elements 670 is shown. An outer layer of criss-cross fibres 672 is provided so as to maintain the inner fibres 674 in the desired position. The inner fibres 674 are a mixture of large 676 and small 678 fibres. By potentially providing the fibres 676, 678 on a number of slightly different alignments a more open structure with large voids is provided. The large gaps in the outer layer of criss-cross fibres 672 means that there is no interference with tissue ingrowth, but these fibres can be provided with a degree of stiffness to assist deployment and positioning of the filling 670 within the space in the bag which surrounds it. A series of lengths of such filling 670 can be used in a single bag to give the desired overall structure.

In FIG. 8, the fibres 685 within a bundle are spaced and provided on a variety of alignments by the inclusion of a number of spherical beads 687.

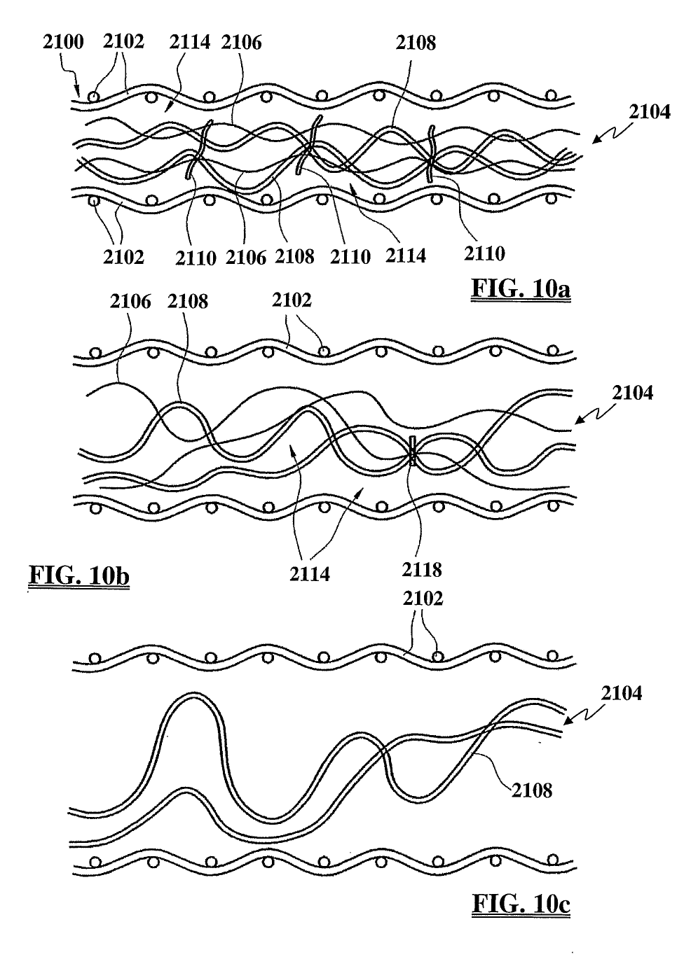

Finally in FIG. 9, a series of beads 690 are provided linked together by a fibre 692. The beads are each surrounded by a mass of fibres 694 braided on to form a mass. The braided mass 694 surrounds each of the beads 690 like a sleeve. Again the filling itself is open and promotes tissue growth.

In many of the above cases, the desired open structure is not only provided by individual groups of fibres, but also by the interaction between individual groups of fibres and the voids between them that they define.

In all of the above embodiments, and in the invention in general, the provision of an open structure can also be assisted by the careful use of different materials for different parts of the filling.

FIG. 10a illustrates a bag 2100 at the time of deployment. The bag 2100 is formed of a number of fibres 2102 woven together to provide the necessary structure for containing filling elements 2104. The filling elements 2104 are provided in the form of a series of wavy fibres of a first size 2106 and second size 2108, together with spacing fibres 2110 which assist in maintaining the open position of the first size 2106 and second size 2108 fibres under compression. The result is an open structure with substantial gaps in the bag 2100 to allow fluid communication through the bag 2100 and substantial voids 2114 between the fibres 2106, 2108, 2110.

Approximately six months (by way of example only) after deployment, as seen in FIG. 10b, the structure of the implant has changed. Substantial amounts of tissue ingrowth has occurred. The tissue ingrowth serves in effect to provide nucleus material which resists compression of the nucleus and filling elements 2104. The spacing fibres 2110 are thus no longer required, having served their function of resisting compression of the fibres 2106, 2108 during the early days of the implant.

By providing the spacing fibres 2110 from a bio-absorbable material which is relatively quickly absorbed, within 6 months (by way of example), the spacing fibres 2110 are removed from the equation. The tissue they served as a scaffold for usefully remains, but the fibres 2110 themselves have degraded in most places. A few remnants 2118 of such fibres 2110 may remain. As a result of these fibres 2110 degrading, there is no restriction on the amount of expansion of voids 2114 formed by the increasing space between fibres 2106, 2108. The tissue growth itself provides the expansive pressure for this to happen.

The non-bioabsorbable fibres 2102 of the bag 2100 remain, as do the fibres 2106, 2108 to provide assistance to the overall structure.

FIG. 10c shows the position approximately 2 years (by way of example only) after deployment. Yet further tissue growth has occurred and the regenerated tissue now provides the majority of the nucleus function. With this mainly biological provision of the necessary structure, there is less need for the fibres 2106, 2108. As the fibres 2106 may also be provided from a bio-absorbable material, these too are may degrade over time. Different time periods for bio-absorption (degradation) may be selected for based on the particular material chosen for use. The degradation of the fibres 2106 allows the remaining fibres 2108 to expand still further.

So as to accurately gauge the size of bag required and amount of filling element needed, it is possible to measure the inflated volume of an inflatable bag inserted into the space vacated by the removed nucleus material.

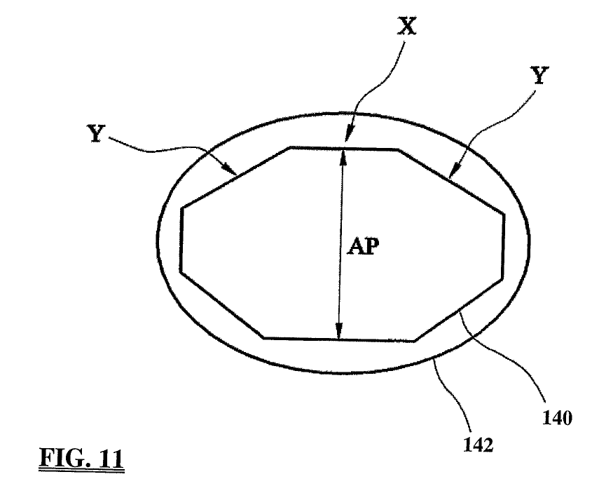

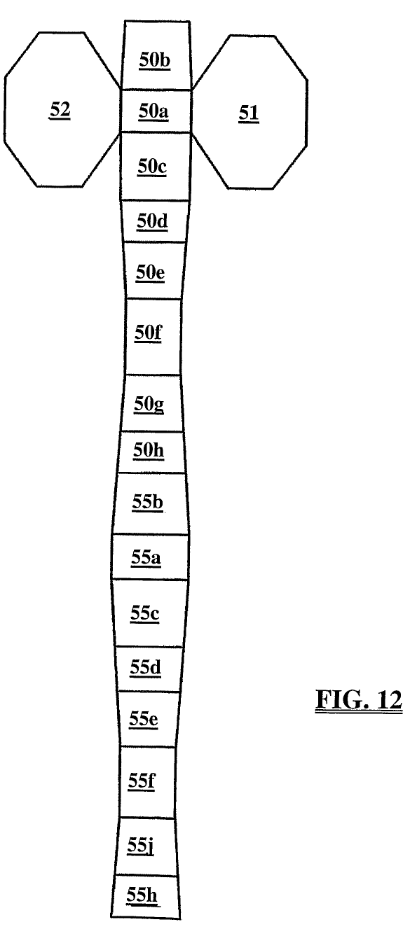

Turning now to FIGS. 11-25, additional embodiments of an implant according to the present invention are shown. These embodiments may provide a more structured void in which to inject filling elements, so as to form the core according to a desired shape or profile. It should be understood that any of the following embodiments may be employed in conjunction with the various filling elements described above, and/or with additional filling elements described below.

A plan profile 140 of an optimised core design is seen in FIG. 11 in comparison with the plan profile 142 of the natural disc it is intended to replace. The naturally curved shape of the disc has been squared off into an octagonal design. This allows easier design of the embroidery element of the disc. Additionally the anterior to posterior length, AP dimension, is reduced compared with the natural disc so as to keep the artificial disc away from the great vessels. When anchoring the device, as described in more detail below, centrally located anchoring on the anterior face, position X, of the vertebrae is avoided, with a preference for anchoring on the adjacent sides, positions Y.

The core could be constructed as a single filling element (e.g. an elongated fiber) or preferably (and particularly where minimally invasive surgery is required), the core may be formed of multiple filling elements which are inserted and assembled to form the overall core in-situ. Such core pieces can be individually inserted and assembled within a single inner jacket, or may be individually wrapped in inner jackets which are then maintained in position by a single outer jacket.

In more varied forms, the core can be formed of potentially tens or hundreds of filling elements comprising small beads. The inner jacket would serve to maintain these in position. A plurality of filling elements formed of elastomer or hydrogel with elastomeric properties are also possible.

Around the core, an inner jacket is provided. This may be embroidered and/or woven. This is separate from a subsequent outer jacket. The inner jacket provides complete encapsulation of the core. As shown in FIG. 12, the jacket is in the form of a first side wall 50a which is connected to a top wall 51 and bottom wall 52. The first side wall 50a is connected to a second side wall 50b in a first direction. In a second direction, the first side wall 50a is connected, in sequence to a third side wall 50c, fourth side wall 50d, fifth side wall 50e, sixth side wall 50f, seventh 50g and eighth 50h. These side wall are stitched to the top wall 51 and bottom wall 52 so as to give an octagonal box form to the inner jacket after when filled.

The material used for the inner jacket uses densely packed fibres to define as smooth a surface as possible for the fabric. This is particularly desirable for the inner surfaces which contact the core. This ensures the most uniform contact surface area between the inner jacket and the core filling.

Connected to the eighth side wall 50h is the first of a series of additional elements also formed from the same embroidery. These additional elements, in sequence 55b, 55a, 55c, 55d, 55e, 55f, 55g and 55h are wrapped around the side walls 50 of the assembled inner jacket. As a result they form an additional ring of material around the side of the core. In effect this extra band of material strengthens the ability of the inner jacket to act as a natural annulus would and resist expansion sideways by the core filling when placed under compressive load. The additional elements can be secured with further stitching. The additional elements 55 could of course be provided by a suitably configured, but separate element to the element providing the walls 51, 52, 50.

The side walls 50 and additional elements 55 are provided with a length and height pattern intended to define an inner jacket which matches the length and height variation of the core shape desired after filling the inner jacket.

An inner jacket provided in this way offers at least two key benefits.

Firstly it allows the jacket in contact with the core to have relatively low movement levels, whilst still enabling the overall desired level of movement for the artificial disc due to the outer jacket's presence and design. Low movement levels for the inner jacket mean that abrasion of the core is minimised. A single jacket would not achieve this.

Secondly, the inner jacket can be designed with properties ideal for its purpose, whilst allowing the outer jacket to be designed with properties ideal for its purpose. Thus the inner jacket aims to provide as dense and hence smooth a fabric surface as possible in contact with the core filling. In this way the risk of individual fibres protruding relative to the others is reduced. Protruding fibres can potentially cause wear due to the micro-motion of the jacket against the core filling in use. This is a particularly relevant issue in the context of the high loads encountered in the lumber region. Whilst such properties are desirable here, they are not consistent with those found to be desirable for the outer surface/outer jacket of the artificial disc. Using two separate jackets allows better optimisation in each case.

In a modified embodiment of the inner jacket, its properties may be tailored to facilitate tissue ingrowth into the space between the inner jacket and the core, and thus also the space between the core filling fibres. The formation of a layer of tissue directly between the jacket and the core of the disc should be beneficial in reducing still further wear in the device. Because the dense fibre form used to provide the most smooth surface contacting the core is not the most conducive to tissue ingrowth, the make up of the inner jacket may be carefully controlled to assist.

By forming the inner jacket with a portion of the fibres or material formed of bio-absorbable material, as tissue ingrowth occurs the inner jacket can be partially absorbed to provide further room for the ingrowth. The non-bioabsorbable material of the inner jacket serves to provide the required structure for the inner jacket over its lifetime, supplemented by the assistance provided by the tissue itself. The use of quickly, moderately and slowly absorbed biomaterials in conjunction with non-absorbable materials can provide a gradual transition from the desired function being provided by the inner jacket alone to the point where it is shared between jacket and tissue. In some cases, an entirely bio-absorbable inner jacket may be provided. Various distributions for the non-absorbable and bio-absorbable material are possible in the inner jacket. The non-absorbable material may particularly form the outside of the inner jacket.

In addition to the core filling and inner jacket, an outer jacket may be provided. A suitable outer jacket is illustrated, by way of example, in FIG. 13. This is intended to substantially surround the inner jacket. The outer jacket has a bottom wall 60 and top wall 62, which are connected by side wall 64a. Further side walls 64b 64c are provided to one side of side wall 64a. Further side walls 64d, 64e are provided to the other side of side wall 64a. Attached to the top wall 62 is a sixth side wall 64f. The top, bottom and side walls are connected to one another by stitching. This leaves two sides of the outer jacket open, in effect the openings defined by edges 66 in one case and 68 in the other.

The edge 66 of the bottom wall 60 is provided with a flange 70. This has a hole 72 in it. The edge 66 of the top wall 62 is provided with a flange 74 which is thinner than flange 70, so as to be able to pass through the hole 72 in flange 70. Similarly, the edge 68 of the bottom wall 60 is provided with a flange 76. This has a hole 78 in it. The edge 68 of the top wall 62 is provided with a flange 80 which is thinner than flange 76, so as to be able to pass through the hole 78 in flange 76. To close the remaining two sides, therefore, flanges 70 and 74 and flanges 76 and 80 are interdigitated.

The flanges 70, 74, 76 and 80 are all significantly longer than the height of the disc space the artificial disc is to be used in. As a result the ends 82 of the flanges 70, 74, 76, 80 can be anchored to the vertebra above the disc replacement in the case of flanges 70 and 76 and to the vertebra below the disc replacement in the case of the flanges 74, 80.

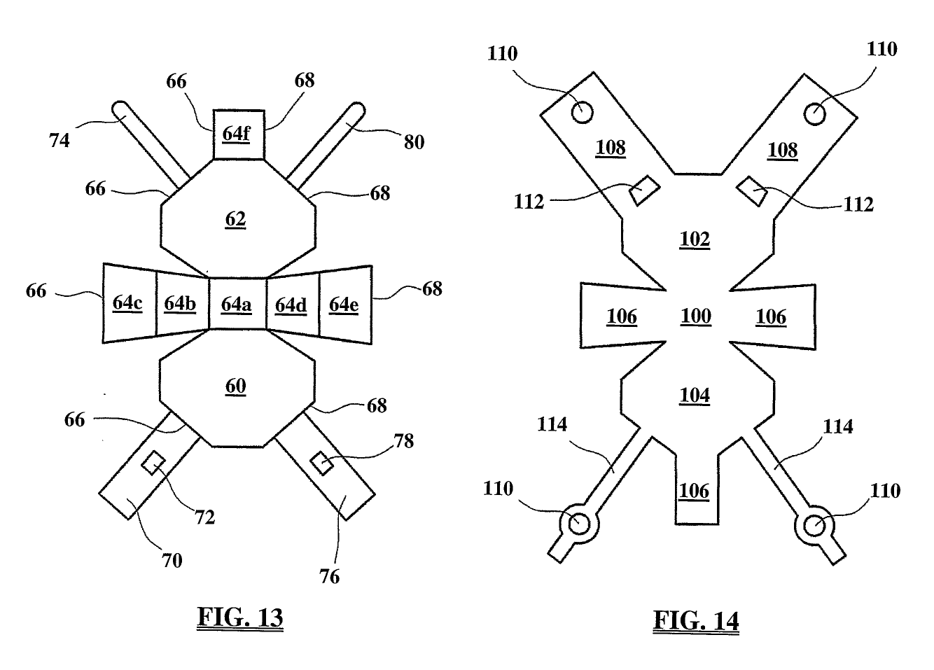

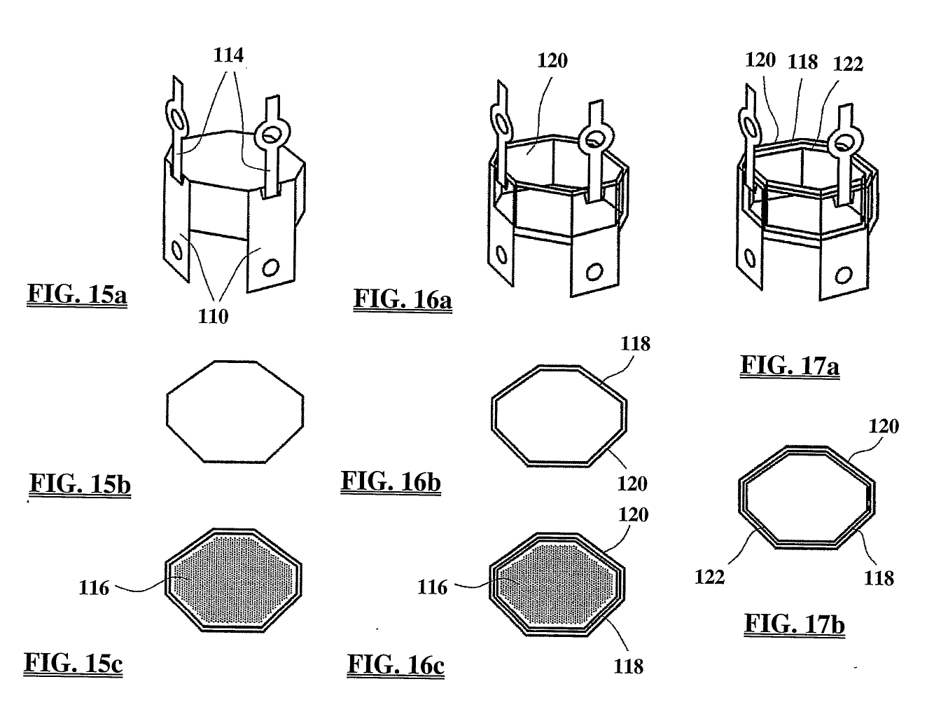

A similar outer jacket to that illustrated in FIG. 13 is provided in FIG. 14. In this case, bottom wall 100 is connected to the top wall 102 by means of side wall 104. Further side walls 106 are provided. Two flanges 108 are provided connected to the top wall 102. These flanges are provided with a hole 110 in each case which is intended to receive the fixing used to collect the device to the spine. These holes are provided towards the ends of the flanges. Close to the top wall 102 two further holes 112 are provided. These have the inner flanges 114 which are connected to the bottom wall 100 passed through them in use, see FIG. 15a. These flanges are also provided with holes 110 to receive fixings in use.

In its assembled form, such a disc outer can appear as shown in FIG. 15a. Here the flanges 114 are clearly shown as interdigitated with the flanges 110 by virtue of their being passed through the holes 112 therein. The completed structure formed by the bottom wall 100, top wall 102, side wall 104 and further side walls 106, together with the flanges, totally encloses the core space. Once again, an octagonal plan view is provided, FIG. 15b, with a similarly shaped octagonal core 116 formed when filled, FIG. 15c, the octagonal core being formed of fibrous filling elements or the like. The core 116 in this case, as with the previous embodiments, is generally centred within the outer jacket.

In the embodiments shown in FIG. 16a, 16b and 16c, an additional ring of material is provided around the core filling, inside the outer jacket 118 by an inner 120. In practice, this provides additional strength to the device when resisting lateral expansion when the core is compressed, i.e. into or out of the paper in the plan view shown in FIG. 16c.

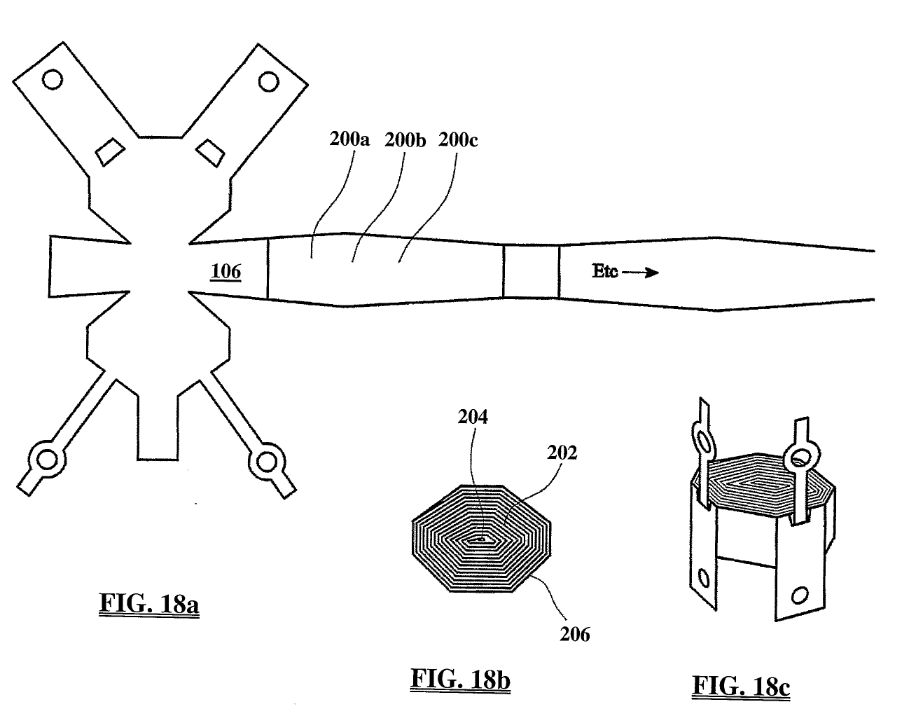

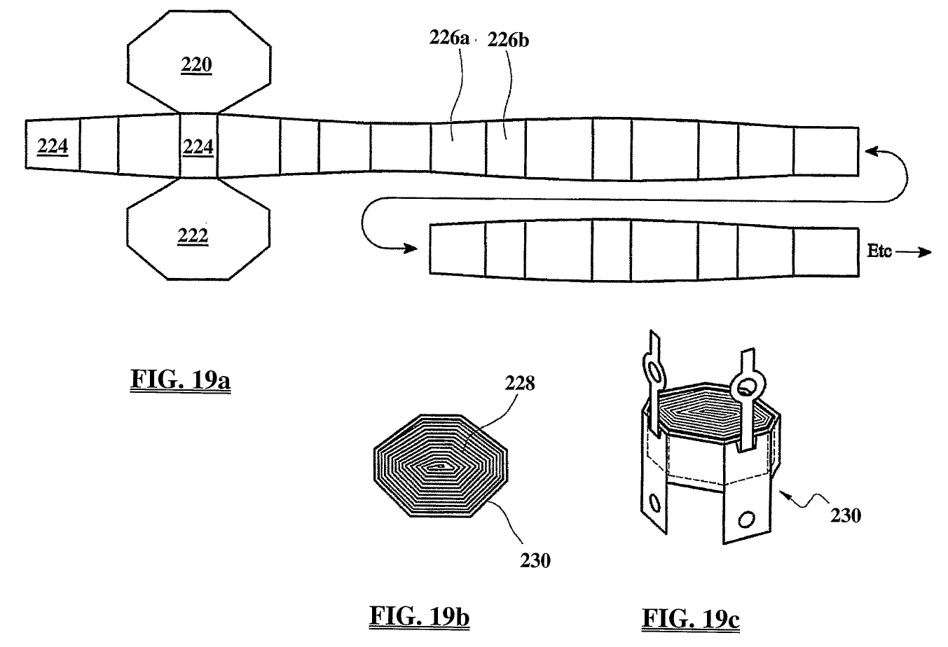

FIG. 18a shows in perspective view the overall assembly consisting of the outer jacket, inner reinforcement and core filling. In this case an additional annular reinforcement 122 is provided.

The embodiment of the invention shown in FIG. 18a provides for a similar outer jacket to that described in FIG. 14 above. However, in this case, the side walls 106 are extended by a very substantial amount via a series of additional elements 200a, 200b, 200c etc. A large number of repeats of these additional elements are provided, a number too great to be shown on the FIG. 18a drawing sheet. This device is assembled by folding the additional elements, starting at one end, so as to form a spiral of generally octagonal outline. The result is shown in FIG. 18b where a spiral 202 is formed extending from the very centre of the device 204, out to its outer wall 206. Such a spiral can be used to fill an inner component and form the core itself, or additional core filling elements can be provided between the turns of the spiral, for instance hydrogel or fibrous material or other filling material which can be caused to flow into the device and then allowed to set. In FIG. 18c, an interdigitated, assembled form of the device of FIG. 18a and FIG. 18b is shown. The spiral core forms the core function for this device, as well as providing substantial reinforcement against expansion when the device is placed under compression. In effect the spiral may provide each of the core, inner component and outer component of the implant in this embodiment.

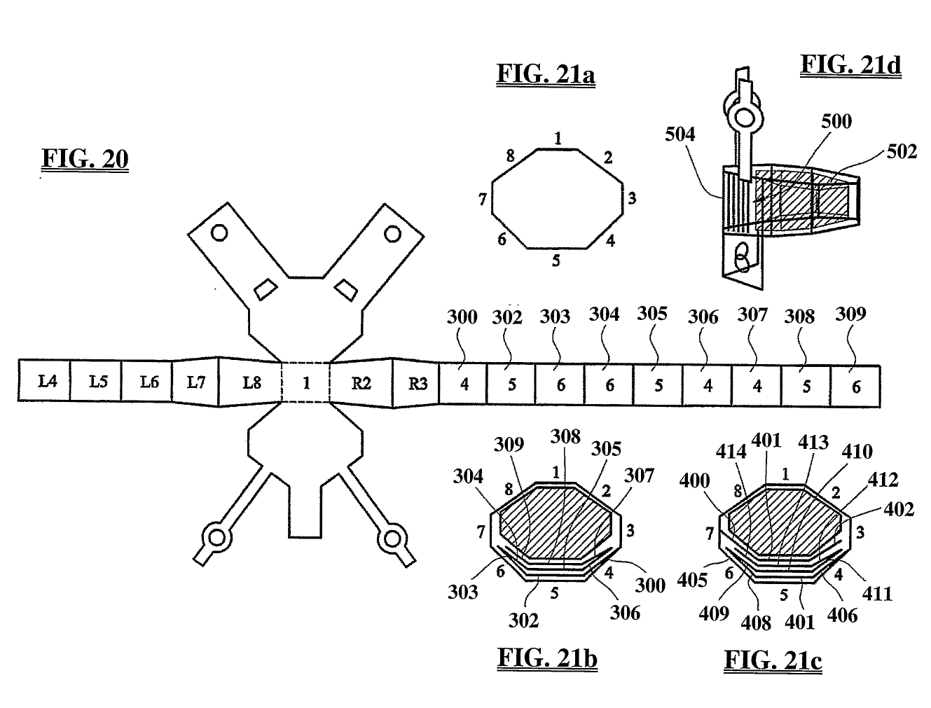

In FIG. 19a, an unassembled form for the inner component is provided, including top wall 220, bottom wall 222, side walls 224 and a large number of additional elements 226a, 226b etc. Once again, these additional elements can be folded so as to provide filling material to fill the exterior 228 of the inner component (comprised of the walls 224, 220 and 222). This in turn is received within an outer component 230, the assembled form for which is shown in FIG. 19c. Again, the folded additional elements may form the core on their own or together with other core filling material, such as hydrogels and/or fibrous material. Again, a core structure of this type provides substantial resistance to sideways expansion when the device is placed under compression. In the FIG. 20 and FIG. 21a to 21d illustrations, a form of device is provided in which the centre of the core is correctly located in the centre of the disc space it is to be provided in. This is achieved by the use of a buttress zone formed in the device. This structure for the device allows the fixation flanges, with their interdigitation, to be flush with the anterior surface of the vertebral bodies, but still allow the disc itself to sit recessed by at least 4 mm within the disc space. Correct centering of the core filling, acting as the replacement, is thus provided. Additionally, such replacement reduces the risk of the main body of the device being pinched by the anterior lip of the vertebrae as the spine is flexed.

Whilst it is possible to form the buttress from an entirely separate component, such as a folded fabric, in the preferred format, it is formed from a series of further elements 300 through to 309. In effect, side walls are provided on the left hand side of the device, as seen in the simple plan view in FIG. 21a by means of the panel L8, L7, L6 and L4. The right hand side is provided by panels R2, R3. The further elements 300 through to 309 are folded to form the buttress structure. A variety of configurations are possible, but in the illustrated form of FIG. 21b, the first part of the buttress is formed by panel 300 which extends inside the outer profile of outer jacket from the edge formed by the contact of panel R3 and L4. Further element 302 extends across the end of panel L5, further element 303 across the inside of panel L6. The further element 304 is then folded back across the inside of further element 303, with further element 305 being across the inside of further element 302. Similarly, further element 306 is provided across the inside of further element 300, before there is a further fold so as to provide further element 307 across the inside of further element 306. Further element 308 is provided across the inside of further element 305 with further element 309 being provided across the inside of the further element 304. Further folds of material can be provided if needed.

An alternative format for the buttress structure, formed in a similar way, is shown in FIG. 21c. Here, further elements provided at one end of the outer jacket form the inner most further elements 400, 401 and 402. Further elements provided between there and the outer wall 405 of the outer jacket are provided by further element 406 through to 414, with further element 414 being the end and lying between further element 400 and further element 409.

A perspective view of such a device, showing the anterior edge 500 of the core 502 recessed relative to the anterior edge 504 of the overall device is shown in FIG. 21d.

The outer jacket has at least three beneficial functions.

Firstly, it provides a jacket against the vertebral end-plates which is separate from the innerjacket that surrounds the core. This reduces micro-motion between the core filling elements and the innerjacket, but still means that the overall level of movement is as desired for the disc replacement as a whole.

Secondly, the outer jacket serves to effectively anchor the artificial disc in place. The interdigitation of the outer jacket effectively retains the inner jacket and core within it. Furthermore, the anchoring for the whole disc achieved through the fixation of the flanges to the vertebrae with screws, bone anchors or a similar type of fixation system is strong. It may be possible, in alternative embodiments to provide a more “free floating” device with the annulus of the disc sutured closed around the device to prevent migration.

Thirdly, the material of the outer jacket can be configured to give the desired structural properties, whilst also providing a relatively open structure for the material. This assists in providing good conditions for tissue ingrowth, both through the outerjacket and eventually through the inner jacket. The outer jacket can provide the desired access, but also act as a scaffold. As with the inner jacket, various combinations of bio-absorbable and non-absorbable materials can be used to assist this process.

The use of an inner jacket and outer jacket is also beneficial in that the use of multiple jackets allows the proportion of embroidery to filling elements (i.e. the core) to remain similar to that established as beneficial in the cervical disc.

In designing the artificial lumbar disc the aim has been to provide a disc having appropriate compressive stiffness. The decompression of the spinal cord through the opening of the disc space is one of the key principles in the relief of pain through disc replacement or fusion. To achieve this the artificial disc is provided with a compressive stiffness curve (force against displacement) similar or higher to the natural disc it is intended to replace. The properties of the core filling elements can be modified by doping or the like. For instance, the filling elements may be provided with 13% barium sulphate.

Ideally, the artificial disc mimics as many of the motion stiffnesses as possible of a natural disc. Flexion/extension motions are both the most common and the largest (in terms of angle) motions that occur in the lumbar spine. This is the key stiffness which the above artificial disc seeks to match. The ability to carry shear and torsional loads on the disc itself should help protect the facet joints and is therefore also mimicked as far as possible.

One of the intentions with disc prostheses of the above mentioned type and type described in U.S. Pat. No. 6,093,205 is to encourage tissue ingrowth into the disc prosthesis. The ingrowth of such soft tissue into the outer jacket and/or inner jacket and/or flanges may occur. The benefit of this is that biological fixation of the prosthesis in the disc space occurs in the long term and this in turn resists undesirable migration of the prosthesis out of the correct position within the disc space. The flanges and the anchoring they provide are particularly useful in this context as they provide secure fixation of the prosthesis whilst this biological fixation develops over the first few months after implantation. The flanges may also provide a useful scaffold for the development of a biological anterior longitudinal ligament.

Whilst the flanges need to provide a high level of fixation during the first few months after implantation, once ingrowth has occurred this level of fixation is not needed. As a result, the level of tension in the flanges needed to give fixation may be undesirably high in the long term as it resists the full extension range of the spine. This is particularly a potential issue for optimum performance in the case of neck disc prostheses, where the extension range is greater.

To address this issue and provide still further improved disc prostheses, designs have been developed which reduce the tension in the flanges a few months after implantation. This may be through a reduction in the tension or its removal through the detachment of the flanges. As a result, once the biological fixation has had time to develop under preferred conditions and with mechanical restraint of the prosthesis, the prosthesis allows the full range of movement and does not compromise the spines operation long term.

A number of designs suitable for general use in the spine, including lumbar and cervical disc spaces have been developed.

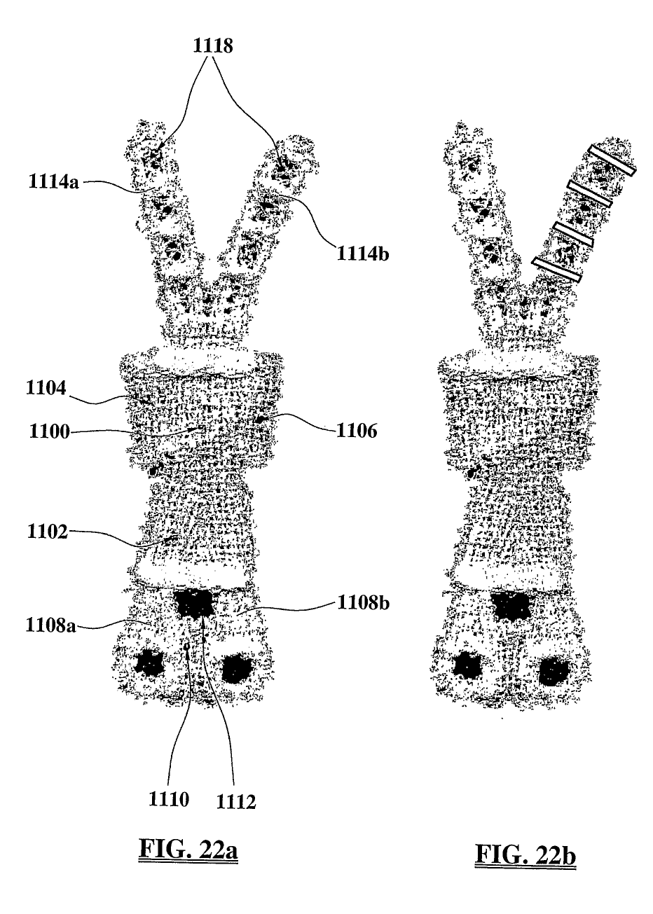

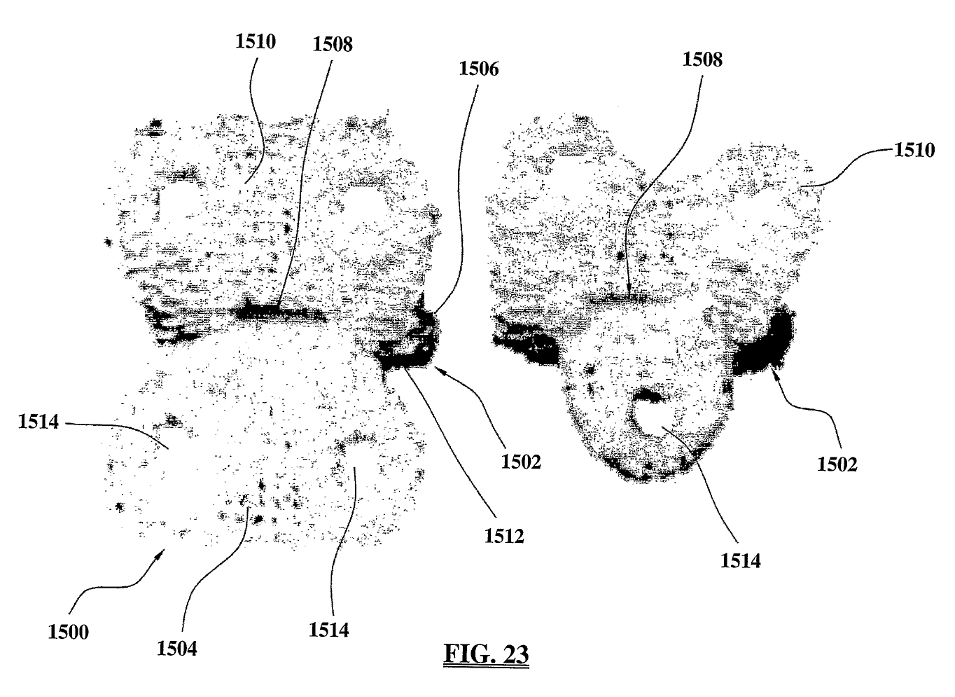

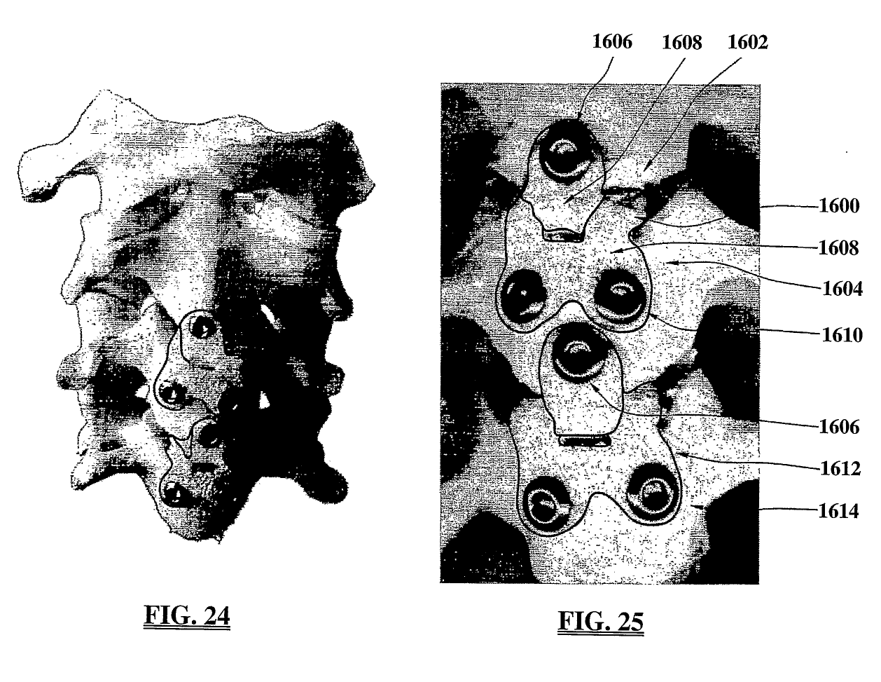

Referring to FIG. 22a, an outer jacket in its flat form is shown, before assembly to allow filling. The core would be surrounded by bottom wall 1100, by the two side walls 1104 and 1106 attached to the bottom wall 1100 and by the top wall 1102. A first pair of flanges 1108a, 1108b extend from the top wall 1102 and are joined together by a web 1110. The web 1110 and flanges 1108a, 1108b define the bounds of a hole 1112. The second pair of flanges 1114a, 1114b are attached to the bottom wall 1100 and in use are passed through the hole 1112 to provide the above mentioned interdigitation. The ends of the flanges 1108a and 1108b both have apertures 1116 which accommodate fixing screws inserted into the spine in use. The ends of the flanges 1114a, 1114b, could be provided with such apertures for fixing screws, but in this case are provided with sections 1118 for receiving sutures, not shown. The operation of this feature is described in more detail below, and of course such a structure could be used in the case of both flange pairs as the fixing.

In a first design approach, the flanges are joined to the rest of the outer jacket which encloses the core filling by a zone of different material. This different material is made of an absorbable fibre and as a consequence, after the desired controlled period, the zone disappears and so ceases to join the flanges to the core filling core (via the outer jacket) anymore. As a result, the tension provided by the flanges is released and the full range of extension is provided. The absorption process would preferably be gradual so as to provide a phase reduction in the tension and hence phased increase in the range of movement.

In a second design approach, the flanges are formed from at least two different material. The flanges include load bearing fibres, which are placed under and maintain the desired tension, and other fibres. The load bearing fibres are made of an absorbable fibre and as a consequence, after the desired controlled period, they are absorbed and so are no longer available to bear the load and the tension is released. The other fibres are intended to be permanent and so are then all that remains of the flanges. These other fibres may serve still to define the overall shape of the flanges, maintain the interdigitation and potentially maintain a reduced level of tension. At least a slackening of the tension results and an increased or even full range of extension is provided. The absorption process would again preferably be gradual so as to provide a phase reduction in the tension and hence phased increase in the range of movement.

In a third design, the flanges include fibres which assume a zigzag path away from the rest of the outer jacket which holds the core and towards the ends of the flanges. When implanted, the zigzag path these fibres take is maintained because these fibres are not subjected to the load applied to the flanges. Instead, that load is borne by other fibres which are attached to the outer jacket and fixation locations. These other fibres are bio-absorbable and so with time disappear. The result is that the load transfers from the other fibres to the zigzag fibres and the zigzag fibres straighten. The result is a slackening of the tension in the flanges and an increase in the range of extension possible.

In a fourth design, the zigzag fibres are again used, but this time together with a series of fibres which bridge the zigzags. The bridging fibres may be stuck to the zigzag fibres and/or wound round them and/or connected to the zigzag fibres in a fixed manner. The overall result is that these bridging fibres prevent the zigzags opening up to a linear form, at the time of implantation, and so prevent the flanges extending, when the desired tension is applied. As the bridging fibres disappear, the load transfers to the zigzag fibres, they straighten, the tension slackens and the extension range for the spine is increased.

In each of these designs, the use of sets of materials in the prostheses means that the transition is made gradual. For instance, slightly different materials and/or different diameters and/or dimensions and/or densities of absorbable material can be used so as to give different periods before each of those different materials is predominantly absorbed and so ceases to bear loads. Slightly different materials could also be used to vary the extent of tissue ingrowth experienced by different parts of the prosthesis, and particularly within different parts of the flanges, between zero and the maximum possible. Zero growth may be desirable where in growth is of no real benefit, for instance in locations where the release of tension would soon render it redundant. Avoiding in-growth in these areas may increase the extent of in-growth where it is beneficial. In-growth may be prevented through the use of appropriate materials to define the fixing locations, for instance. Ultra-high molecular weight polyethylene may be used as such a material.

The ends of the flanges, as mentioned briefly above, are provided with sections 1118 for receiving sutures. Such an arrangement could be provided for the ends of both pairs of flanges. These sections are formed of a reinforced parts 1120 which extend across the flanges between the load bearing fibres 1122 on one side of the flange and the load bearing fibres 1122 on the other side of the flange. A series of such reinforced parts 1120 are provided spaced along the length of the flanges. Between the reinforced parts 1120 are mesh parts 1124 forming openings which are criss-crossed by a series of fibres. These mesh parts 1124 allow the suture to be readily positioned by wrapping it around the reinforced parts 1120. By providing a series of alternating mesh parts 1124 and reinforced parts 1122 along the flanges a variety of fixing locations for use in attaching to the spine are provided.

FIG. 23 shows on the left hand side, an outer jacket 1500 of one form of the present invention. The body 1502 of the outer jacket 1500 surrounds the filling elements comprising the core. The flange 1504 extending from the top surface 1506 of the body 1502 passes down through a hole 1508 in the flange 1510 extending from the bottom surface 1512 of the body 1502. The resulting interdigitation closes off the opening in the body 1502 which allows the core to be introduced. Each flange 1504, 1510 is provided with two holes 1514 which receiving fixings to attach the flanges to the spine.

In an another form, shown on the right hand side of FIG. 23, the body 1502 and lower flange 1510 extending from it are provided in the same way as the left hand side form described above. The difference lies in the configuration of the other flange 1520. Again this flange 1520 is interdigitated with the flange 1510 by being passed through a hole 1508 in the flange 1510. The flange 1520 is provided with a single hole 1514 which receives a fixing. However, the flange 1520 does not flare out to as great a width as the flange 1504 in the left hand side form. This results in a generally Y-shaped profile presented by the parts of the flanges 1510, 1520 extending beyond the location of interdigitation.

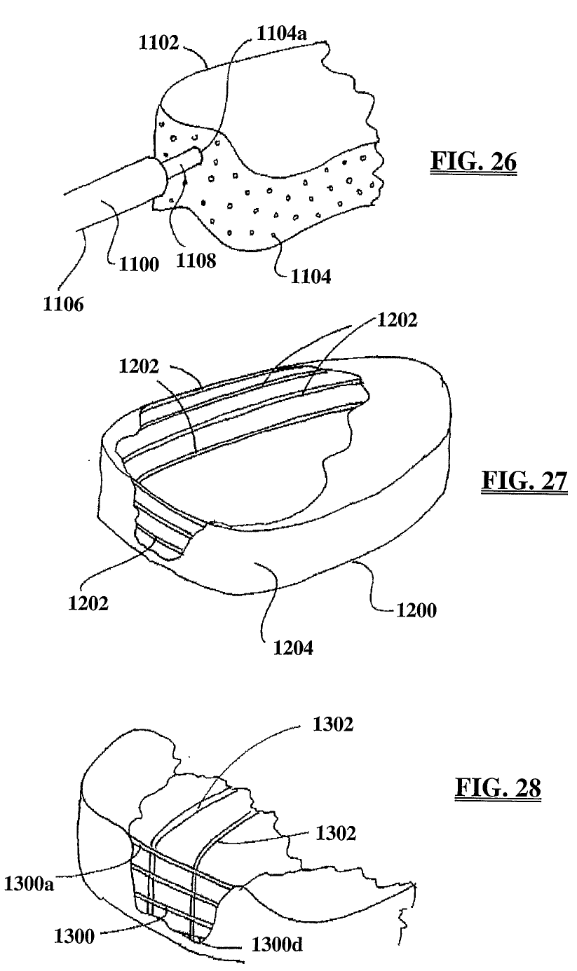

The benefits of the Y-shaped profile are explained with reference to FIG. 24 and FIG. 25. One assembled artificial disc 1600 is inserted between a first vertebra 1602 and a second vertebra 1604. The artificial disc 1600 is fixed to the first vertebra 1602 by virtue of a fixing 1606 which passes through the hole in the flange 1608. The head of the fixing 1606 is larger than the hole in the flange 1608 it passes through so giving a secure fixing to the vertebra 1602. The artificial disc 1600 is fixed to the second vertebra 1604 by virtue of two fixings 1610. Thus the stem of the Y-shaped profile is fixed to the first vertebra 1602, whilst the fork of the Y-shaped profile is fixed to the second vertebra 1604.

A second assembled artificial disc 1612 is inserted between a third vertebra 1614 and the second vertebra 1604. The second artificial disc 1612 is provided with the Y-shaped profile in the same orientation. Thus the fork of the Y-shaped profile is fixed to the third vertebra 1614, whilst the stem of the Y-shaped profile is fixed to the second vertebra 1604. This means that the second vertebra 1604 need only accommodate one fixing 1606 from the second artificial disc 1612 and two from the first artificial disc 1600, with those fixings in different positions across the face of the second vertebra 1604. This means that the fixings take up less room because of the lower number used, at even less room because of the different positions they occupy. The central fixing 1606 of the second artificial disc 1612 can be nested between the fixings 1610 of the first artificial disc 1600.

The nesting or interlocking nature of disc flanges provided in this way enable artificial discs to be provided at adjacent levels along this spine. This arrangement is particularly useful in the context of the cervical part of the spine where space is limited. As well as using a reduced number of fixings, this form of flanges also avoids overlapping of the flange from one disc replacement with the flange of another. Overlapping material is undesirable as it increases the space occupied by the replacement disc on the anterior face of the spine and renders the replacement less minimal. The flanges of the disc replacement still provided the desired anterior longitudinal ligament replacement. The fixings still provide the desired torsional stability. This type of artificial disc is still useful where only a single disc replacement is needed, however.

FIG. 26 illustrates a further embodiment of the invention wherein the filling elements may be introduced into (by way of example only) the outer component 1102 via a filling instrument 1100. In this instance, the outer component 1102 is not provided with an opening in an end, but instead is filled through one of the pores 1104 in the outer component 1102. The filling instrument 1100 may be provided as an injection tool 1106 having a tip 1108 of reduced diameter dimensioned to be pushed into a pore 1104a. In doing so, the size of the pore 1104a is increased by stretching to be greater than the size of the tip 1108. The filling elements (e.g. one or more filaments as described above) can then be injected. The density of the filling element(s) is such that it/they can readily flow under pressure from the injection tool 1106 through an opening of size 1104a. However, the density is such that the filling elements cannot readily flow through an opening of size 1104, particularly when under the lower pressure levels experienced within the outer component 1102 compared with those experienced in the tip 1108. The same principle would apply where the filling includes distinct particles such as beads. The stretched pore is large enough to allow the filling in, but the normal size pores are too small to allow the filling out.

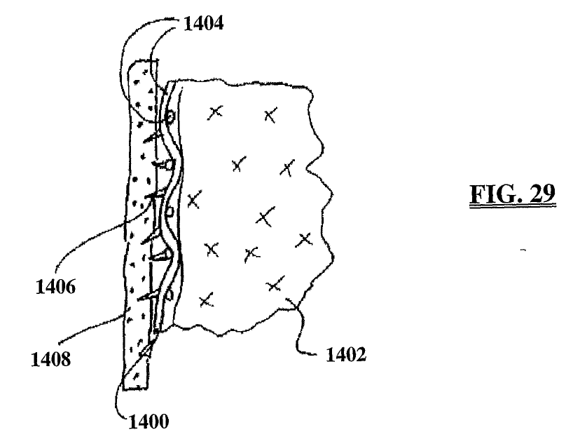

Whilst the inner and/or outer component can be entirely flexible, consistent with its fabric/textile nature, there are benefits in providing a more defined structure or profile to one or both of these components. Thus, as shown by way of example with regard to outer component 1200 in the embodiment of FIG. 27, the outer component 1200 is provided of fabric, but within the bag a number of stiffening elements 1202 are provided. Thus a series of stiffening elements 1202a are provided in the form of rings which extend around the periphery of the outer component 1200 and so seek to maintain the side wall 1204 profile of the outer component 1200. For insertion, the sides of the rings can be squeezed together and so reduce the cross-section of the outer component 1200. Once inside the disc space, the compression can be removed and the rings will push the sides 1204 of the outer component 1200 outwards to the disc like profile. This assists in ensuring the shape of the implant is correct and assists in providing the space into which the filling elements can be introduced.

In the FIG. 28 detail, the stiffening elements 1300 are supplemented by stiffening elements 1302 which seek to push the top 1300a and bottom 1300d rings apart in a vertical sense. Again a downward compression can be used to reduce the profile of the outer component 1300, with the removal of that compression allowing the outer component 1300 to return to the desired form.

Such arrangements of stiffening elements can be used to close or assist in supporting the closure of the inner and/or outer component. Equally such stiffening elements can be used to support surfaces of the inner and/or outer component against loads. For instance, the surface of the implant which faces the vertebra above the implant in a standing individual and/or the surface which faces the vertebra below may be provided with stiffening elements which extend across them to resist loads. Stiffening elements down the sides, round the edges and at other positions may also be provided to support the shape of the implant and/or contribute to its functional characteristics. Resistance to load, extension, compression, flexion or the like may be provided in this way, as might resistance to tissue ingrowth pressures.

The characteristics provided by the stiffening elements may be different for different parts of the implant. For instance, some parts may be less resistant to a force than others. Metal wires, metal fibres, stiff plastics wires or fibres and the like could be used for the stiffening elements. In particular shape memory materials, such as nitinol, could be used for the stiffening elements. A wide variety of configurations are possible, including rings, spirals, zig-zags, loops, coils, waves and others.

In the embodiment illustrated in FIG. 29, the detailed view shows a cross-section through (by way of example only) the outer component 1400 and filling 1402 which in this case is a bundle of one or more filaments or fibres. The fibres 1404 which are woven together to form the outer component 1400 include on their outside a series of projections 1406. These projections 1406 are integrally formed with the fibres 1404 and are provided at an inclined angle. As such as the outer component 1400 expands during filling and/or moves during insertion/filling/use, the projections 1406 act as barbs and dig into the surrounding material of the annulus 1408. In this way, a firm anchorage for the implant is provided all over its surface, including those parts which could not be reached from the small incision used to insert the implant. If sutures or staples are to be used to fix the implant within the annulus, then they can only really be provided at or close to the incision site.

While this invention has been described in terms of a best mode for achieving this invention's objectives, it will be appreciated by those skilled in the art that variations may be accomplished in view of these teachings without deviating from the spirit or scope of the present invention.

1. A disc prosthesis comprising:
a core including at least one filling element;
an inner fabric component completely encapsulating the core and including a smooth inner contact surface configured to interface with the core, and
an outer fabric component dimensioned to at least partially encapsulate the inner fabric component;
wherein movement between the inner and outer fabric components is facilitated in preference to movement between the inner fabric component and core.

2. (canceled)

3. The disc prosthesis of claim 1, wherein the at least one filling element is fibrous.

4. The disc prosthesis of claim 1, wherein the at least one filling element comprises a porous component, the porous component defined by at least one of pores, voids, apertures, and gaps formed at least one of within the at least one filling element, between the at least one filling element at least one other filling element, and between parts of the at least one filling element.

5. The disc prosthesis of claim 3, wherein the at least one filling element is formed of at least one of unconstrained fibers, unbraided fibers and, interlaced fibers.

6. The disc prosthesis of claim 1, wherein the core further comprises a top surface and a bottom surface, and at least one of the top surface and bottom surface of the core is at least one of octagonal, hexagonal, round, and elliptic.

7-8. (canceled)

9. The disc prosthesis of claim 1, wherein the inner fabric component is at least one of configured and formed with at least one material intended to promote tissue growth.

10. The disc prosthesis of claim 1, wherein at least one material used in at least one of the inner fabric component, the outer fabric component, and the filling element is bio-absorbable.

11. The disc prosthesis of claim 1, wherein the interface between the inner contact surface of the inner fabric component and the core comprises a uniform contact.

12-13. (canceled)

14. The disc prosthesis claim 1, wherein the inner fabric component further comprises a fabric extension forming a continuous band extending at least partially around the side of the inner fabric component.

15. (canceled)

16. The disc prosthesis of claim 1, wherein the outer fabric component is at least one of configured and formed of at least one material intended to promote tissue growth, particularly tissue in-growth at least one of through the outer fabric component, between the inner fabric component and the core, and through the inner fabric component.

17-18. (canceled)

19. The disc prosthesis of claim 1, wherein the outer fabric component includes at least one flange extending therefrom, the at least one flange providing at least one anchor location for attaching the outer fabric component to at least one vertebra.

20-21. (canceled)

22. A method of performing spine surgery, the method comprising:
providing a disc prosthesis comprising:
a core including at least one filling element;
an inner fabric component completely encapsulating the core and including a smooth inner contact surface configured to interface with the core; and
an outer fabric component dimensioned to at least partially encapsulate the inner fabric component;
removing at least part of a natural disc from an intervertebral space within a spine; and
inserting the disc prosthesis into the intervertebral space formerly occupied by the removed natural disc.

23. (canceled)

24. The disc prosthesis of claim 1, wherein the inner fabric component is at least one of a bag and other form of container having at least one of an opening and pore formed therein to permit the insertion of the at least one filling element.

25. The disc prosthesis of claim 4, wherein the pores in the porous component have at least one cross-sectional dimension that is less than the smallest cross-sectional dimension of the filling elements.

26-32. (canceled)

33. The disc prosthesis of claim 3, wherein the at least one filling element is provided with at least one of aligned, wavy, curved, and zigzag fibers.

34-35. (canceled)

36. The disc prosthesis of claim 3, wherein the at least one filling element includes fibers having at least two different cross sections.

37. The disc prosthesis of claim 3, wherein the at least one filling element includes a plurality of fibers provided in a first direction and further includes at least one of a restraining fiber and material which at least one of surrounds, encloses, wraps around, and contacts the plurality of fibers.

38. The disc prosthesis of claim 3, wherein the at least one filling element includes at least one of peripheral fibers and material provided around the filling element, the at least one of peripheral fibers and material being wrapped around the at least one filling element in at least one of a spiral manner and crisscross manner.

39. The disc prosthesis of claim 3, wherein the at least one filling element includes at least one of spheres, beads, and blocks intermixed with at least one fiber.

40-41. (canceled)

42. The disc prosthesis of claim 4, wherein the at least one filling element is at least one of configured and formed of at least one material intended to promote tissue growth, particularly tissue ingrowth at least one of through at least one filling element, between the porous component and at least one filling element, and between two or more filling elements.

43. (canceled)

44. The disc prosthesis of claim 10, wherein the bio-absorbable material restrains at least part of the at least one filling element in a first state, the bio-absorption of the material allowing at least part of the at least one filling element to assume a second state, the second state providing for at least one of a greater internal volume of at the least one filling element, greater porosity for the at least one filling element, reduction in mass of the at least one filling element, and more space for tissue in-growth.

45-47. (canceled)

48. The disc prosthesis of claim 1, wherein at least one of the inner fabric component and outer fabric component is provided with at least one stiffening element.

49. The disc prosthesis of claim 48, wherein the at least one stiffening element is comprised of at least one of metal wire, metal fiber, stiff plastic wire, and shape memory material, and wherein the at least one stiffening element is shaped in at least one of a ring, spiral, zigzag, loop, coil, and wave.

50. The disc prosthesis of claim 1, wherein the outer fabric component includes a plurality of projections configured to extend into adjacent tissue when the disc prosthesis is implanted within an intervertebral space.

51. A disc prosthesis comprising:
a core including at least one filling element;
an inner fabric component completely encapsulating the core and including a smooth inner contact surface configured to interface with the core;
an outer fabric component dimensioned to at least partially encapsulate the inner fabric component; and
at least one fabric flange member extending from the outer fabric component, the flange member including at least one anchor location for anchoring the disc prosthesis to at least one vertebra, the flange member further including a bio-absorbable component, the bio-absorbable component configured to gradually absorb over time such that upon absorption, the disc prosthesis will allow extension motion.

52. The disc prosthesis of claim 51, wherein the bio-absorbable component comprises a zone of bio-absorbable fabric separating the flange from the outer fabric component, the bio-absorbable fabric configured to gradually absorb over time such that upon absorption, the flange member is no longer attached to the outer fabric component and the disc prosthesis will allow extension motion.

53. The disc prosthesis of claim 51, wherein the bio-absorbable component comprises a plurality of bio-absorbable load-bearing fibers configured to gradually absorb over time such that upon absorption, the flange member exhibits at least a partial slackening of tension and the disc prosthesis will at least partially allow for extension motion.

54. The disc prosthesis of claim 53, wherein the flange member further includes a plurality of non-absorbable fibers formed in a zigzag path from the outer fabric component to a distal end of the flange member such that upon absorption of the bio-absorbable component, the non-absorbable fibers will straighten to limit the range of extension possible.

55. The disc prosthesis of claim 51, wherein the time need for absorption of the bio-absorbable component is customizable by varying at least one of the material, diameter, dimensions, and densities of the bio-absorbable component.

56. A method of performing multi-level spinal fusion, comprising:
providing a first disc prosthesis including a body and first and second flange members, the body comprising a core including at least one filling element and an outer fabric component dimensioned to encapsulate the core, the first flange member including first and second anchoring locations dimensioned to receive an anchor element, the second flange member including a third anchor location dimensioned to receive an anchor element;
providing a second disc prosthesis including a body and first and second flange members, the body comprising a core including at least one filling element and an outer fabric component dimensioned to encapsulate the core, the first flange member including first and second anchoring locations dimensioned to receive an anchor element, the second flange member including a third anchor location dimensioned to receive an anchor element;
implanting the first disc prosthesis by inserting the body into a space between a first vertebral body and a second vertebral body, affixing the first flange member of the first disc prosthesis to the first vertebral body, and affixing the second flange member of the first disc prosthesis to the second vertebral body; and
implanting the second disc prosthesis by inserting the body into a space between the second vertebral body and a third vertebral body, affixing the first flange member of the second disc prosthesis to the second vertebral body, and affixing the second flange member of the second disc prosthesis to the third vertebral body.

57. The method of claim 56, wherein the second flange member of the first disc prosthesis is affixed to the second vertebral body at a location at least partially between the first and second anchor locations of the first flange of the second disc prosthesis.

58. The method of claim 56, wherein the first flange member of the second disc prosthesis is shaped to include a gap between the first and second anchor locations located thereon.

59. The method of claim 58, wherein the second flange member of the first disc prosthesis is shaped to nest within the gap such that the second flange member of the first disc prosthesis and the first flange member of the second disc prosthesis do not overlap.

60. A system for multi-level spinal fusion, comprising:
a first disc prosthesis including a body and first and second flange members, the body comprising a core including at least one filling element and an outer fabric component dimensioned to encapsulate the core, the first flange member including first and second anchoring locations dimensioned to receive an anchor element, the second flange member including a third anchor location dimensioned to receive an anchor element, the first and second flange members extending from the outer fabric component such that the first flange member is positionable over a first vertebral body and the second flange member is positionable over a second vertebral body; and
a second disc prosthesis including a body and first and second flange members, the body comprising a core including at least one filling element and an outer fabric component dimensioned to encapsulate the core, the first flange member including first and second anchoring locations dimensioned to receive an anchor element, the second flange member including a third anchor location dimensioned to receive an anchor element, the first and second flange members extending from the outer fabric component such that the first flange member is positionable over the second vertebral body and the second flange member is positionable over a third vertebral body.

61. The system of claim 60, wherein the first flange member of the second disc prosthesis is shaped to include a gap between the first and second anchor locations located thereon.

62. The system of claim 61, wherein the second flange member of the first disc prosthesis is shaped to nest within the gap such that the second flange member of the first disc prosthesis and the first flange member of the second disc prosthesis do not overlap.
